(19) United States
(12) Patent Application Publication
(10) Pub. No.: US 2024/0331798 A1
(43) Pub. Date: Oct. 3, 2024

(54) INTERPRETABLE RNA FOUNDATION MODEL FOR RNA STRUCTURE AND FUNCTION PREDICTIONS

(71) Applicant: The Chinese University of Hong Kong, Shatin (CN)

(72) Inventors: Yu LI, Shatin (CN); Jiayang CHEN, Fanling (CN)

(73) Assignee: The Chinese University of Hong Kong, Shatin (CN)

(21) Appl. No.: 18/619,010

(22) Filed: Mar. 27, 2024

Related U.S. Application Data
(60) Provisional application No. 63/455,134, filed on Mar. 28, 2023.

(51) Int. Cl. G16B 15/10; G16B 40/20; G16B 40/30

(52) U.S. Cl. CPC G16B 15/10; G16B 40/20; G16B 40/30

(57) ABSTRACT

A foundation model for analysis of RNA sequences, including ncRNA sequences, can be trained to provide output embeddings (in a high-dimensional space) corresponding to input RNA sequences. Training of the RNA foundation model can use a large-scale dataset of RNA sequences without any annotation as to structure or function. The trained RNA foundation model can thereafter be used to produce embeddings that can be used as input features in downstream task-specific machine-learning models (or other computer models) that can learn to predict particular aspects of structure and/or function for a given RNA sequence.

### CROSS-REFERENCE TO RELATED APPLICATION

This application claims priority to U.S. Provisional Application No. 63/455,134, filed Mar. 28, 2023, which is incorporated herein by reference.

### BACKGROUND

RNA (ribonucleic acid) plays an important role in performing numerous biological functions, such as cell signaling, gene expression, and post-transcriptional regulation. Determination of RNA structure or function is also an important aspect of RNA-based therapeutics. Among all RNA transcripts, only about 5% serve as messenger RNAs (mRNAs) responsible for protein coding, while the substantial remaining portion is non-coding RNAs (ncRNAs). These ncRNAs sustain specific structures to conduct corresponding biological functions, playing an important role in controlling and regulating biological activity. Although many ncRNA sequences are currently known, structure and/or function is known for few of them. Computational methods for predicting structure and/or function suffer due to the scarcity of annotated data.

Existing approaches to RNA structure prediction focus on prediction of RNA secondary structure. Such approaches can be further divided into three categories: thermodynamic methods, alignment-based methods, and deep learning (or “DL”)-based methods. Thermodynamic methods date to the 1980s and may have reached a plateau, which may be in part because they usually do not consider all base pairs obtained from tertiary interactions and therefore may miss important information. Alignment-based methods build upon comparative sequence analysis and are designed to determine vital base pairs among homologous sequences. However, effectiveness of this approach has been limited by the limited number of known RNA families. DL-based approaches seek to overcome these limitations, but existing model architectures are explicitly designed for a particular task and do not generalize well to unknown RNA types.

In contrast to secondary structure prediction, modeling of three-dimensional (3D) structure of RNA is under-explored, due in part to the lack of 3D structure data. Some efforts have been made to optimize 3D structure based on minimum energy given 2D information, where the 2D information (secondary structure, distance structure) can be obtained using deep learning methods. However, there are no end-to-end DL-based methods that can generate RNA 3D structure directly. This is in part due to the lack of annotated 3D data.

Understanding the function of specific RNAs (particularly ncRNAs) is also desirable. For instance, predicting interactions between RNAs and proteins may assist in understanding regulation of gene expression. Existing databases provide hand-coded labeling that classifies known RNAs into several groups based on biological experiments. DL-based approaches have been proposed for learning the underlying distribution of RNAs in different functional groups, which (in theory) could enable prediction of functional group for a new RNA. However, because these approaches rely on hand-annotated information about RNA sequences, the ability to generalize is limited.

### SUMMARY

Certain embodiments of the present invention relate to training and utilization of a language model (or transformer model) for RNA sequences (including ncRNA). The model can be trained using a large-scale dataset of RNA sequences without any annotation (e.g., as to structure or function). This model, referred to herein as an RNA “foundation” model (or “RNA-FM”) can receive an RNA sequence as input and produce an output embedding (referred to herein as an “RNA-FM embedding”). RNA-FM embeddings be used in training of downstream task-specific neural networks or other machine learning models that can learn to predict particular aspects of structure and/or function for a given RNA sequence. For example, a downstream neural network can be trained to predict secondary structure of ncRNA or to predict RNA-protein interactions using the RNA-FM embeddings (and optionally the original RNA sequences) as input features.

Some embodiments relate to computer-implemented methods for providing an RNA foundation model. Such methods can include: obtaining a large-scale training dataset of RNA sequences including unannotated RNA sequences; training an RNA foundation model using the large-scale training dataset, wherein the RNA foundation model includes a plurality of transformer encoder blocks that produce an output embedding corresponding to an input RNA sequence; and providing a query interface to the trained RNA foundation model, wherein the query interface receives a query RNA sequence and produces a corresponding output embedding. In some embodiments, the RNA foundation model includes an initial embedding layer that embeds each nucleotide token into a high-dimensional vector.

In various embodiments, training of the RNA foundation model can be performed using a self-supervised training process. For example, the training process can include: randomly replacing a fraction of original nucleotide tokens in a first RNA sequence from the large-scale training dataset with either a mask token or a randomly-selected nucleotide token to produce a masked sequence; using the RNA foundation model to generate an output embedding for the masked sequence; and predicting, based on the output embedding for the masked sequence, which original nucleotide token corresponds to a particular mask token in the masked sequence. In some embodiments, the training process can further include computing a cross-entropy loss based at least in part on the prediction.

In various embodiments, a large-scale training data set can be obtained by mining existing RNA databases to obtain an initial dataset of RNA sequences. In some embodiments, the initial dataset can consist of or include non-coding RNA (ncRNA) sequences. Preprocessing can be applied to obtain the large-scale training dataset; examples of preprocessing include standardizing nucleotide tokens and removing duplicate RNA sequences.

In various embodiments, a task-specific downstream system can be trained to predict a structural or functional characteristic of an input RNA sequence. The task-specific downstream system can include a module that uses the query interface of the trained RNA foundation model to obtain an output embedding corresponding to the input RNA sequence and a machine-learning module that uses the input RNA sequence and the corresponding output embedding as inputs. Training of the task-specific downstream system can include supervised, unsupervised, or semi-supervised training processes as desired. For example, a task-specific downstream system may be trained to predict secondary structure of a given RNA sequence. It should be understood that multiple different task-specific downstream systems can be trained to predict different structural or functional characteristics and wherein all of the task-specific downstream systems obtain output embeddings from the same trained RNA foundation model.

Some embodiments relate to computer-implemented methods for predicting various aspects related to structure or function of an RNA sequence using a trained RNA foundation model. For each of a plurality of RNA sequences, a corresponding output embedding can be obtained from an RNA foundation model that includes a plurality of transformer encoder blocks and that has been pre-trained to produce an output embedding corresponding to an input RNA sequence using an unsupervised learning process. A task-specific machine-learning model can be trained to predict a structural or functional characteristic of an input RNA sequence (e.g., using a supervised learning process with annotated training data), where the task-specific machine-learning model uses as input a combination of the input RNA sequence and the corresponding output embedding produced by the RNA foundation model. The trained task-specific machine-learning model can be used to make a prediction for a testing input RNA sequence. For example, a task-specific machine-learning model can be trained to predict secondary structure of the input RNA sequence. As another example, a task-specific machine-learning model can be trained to predict a protein-RNA interaction of the input RNA sequence or a parameter related to a gene expression regulation function of the input RNA sequence.

Some embodiments relate to computer systems that can train and/or host an RNA foundation model for use by downstream systems or processes. For example, a computer system can include a memory to store an RNA foundation model that includes a plurality of transformer encoder blocks and that has been pre-trained to produce an output embedding corresponding to an input RNA sequence using an unsupervised learning process; an interface to receive queries from one or more requesting systems, each query including a queried RNA sequence; and a processor coupled to the interface and the memory. The processor can be configured (e.g., using suitable program code) to: input the queried RNA sequence into the RNA foundation model to obtain a corresponding output embedding; and return the output embedding to the requesting system via the interface. In some embodiments, the processor can also be configured to train the RNA foundation model. Requesting systems can use the output embedding in a machine-learning task that predicts a structural or functional characteristic of the queried RNA sequence based at least in part on the output embedding.

The following detailed description, together with the accompanying drawings, will provide a better understanding of the nature and advantages of the claimed invention.

### BRIEF DESCRIPTION OF THE DRAWINGS

FIG. 1 show a flow diagram of a process for training and using an RNA foundation model according to some embodiments.

FIG. 2 shows an overview of a system for training an RNA foundation model according to some embodiments.

FIG. 3 shows an overview of a system for downstream use of an RNA foundation model according to some embodiments.

FIGS. 4A-4C show graphs of results obtained from an RNA atlas constructed using RNA-FM embeddings according to an embodiment.

FIG. 5 shows a table summarizing results of a study of secondary structure prediction using RNA-FM embeddings according to an embodiment.

FIGS. 6A and 6B show graphs comparing the performance of secondary structure prediction using RNA-FM embeddings according to an embodiment and using a conventional approach.

FIGS. 7A and 7B show two examples of binary probability maps predicted by secondary structure prediction models using RNA-FM embeddings according to an embodiment and using a conventional approach.

FIG. 8 shows a table comparing precision of long-range structure prediction from prediction models using RNA-FM embeddings according to an embodiment and conventional prediction models.

FIGS. 9A-9C show graphical comparisons of the performance of long-range structure prediction from prediction models using RNA-FM embeddings according to an embodiment and conventional prediction models.

FIG. 10 shows a table summarizing 3D distance prediction performance from prediction models using RNA-FM embeddings according to an embodiment and conventional prediction models.

FIGS. 11A-11C show graphical illustrations comparing 3D distance prediction performance from prediction models using RNA-FM embeddings according to an embodiment and conventional prediction models.

FIGS. 12 and 13 illustrate examples of reconstructing a 3D RNA structure based on prediction models using RNA-FM embeddings according to an embodiment and conventional prediction models.

FIG. 14A shows a schematic diagram representing SARS-CoV-2 and indicating sampled segments used in a study described herein.

FIG. 14B shows violin plots of performance parameters for predicting secondary structure for SARS-CoV-2 based on prediction models using RNA-FM embeddings according to an embodiment and conventional prediction models.

FIG. 14C shows a visualization of RNA secondary structure predictions in the 5′UTR region of SARS-CoV-2 based on a prediction model using RNA-FM embeddings according to an embodiment.

FIG. 15A shows a phylogenetic tree representing ground truth for the evolutionary trend of COVID-19.

FIG. 15B shows a streamplot and associate projections for the evolutionary trajectory of COVID-19 as inferred using RNA-FM embeddings according to an embodiment.

FIG. 16 shows a schematic illustration of a deep learning model for RNA-protein interaction modeling that can be enhanced using RNA-FM embeddings according to an embodiment.

FIG. 17 shows a table summarizing results obtained from the deep learning model of FIG. 16 using RNA-FM embeddings according to an embodiment and conventional inputs.

FIGS. 18A and 18B show graphical representations of results obtained from the deep learning model of FIG. 16 using RNA-FM embeddings according to an embodiment and conventional inputs.

FIG. 19 shows a schematic illustration of a deep learning framework for predicting mean ribosome load from RNA sequences that can be enhanced using RNA-FM embeddings according to an embodiment.

FIG. 20 shows a table summarizing results obtained from the deep learning framework of FIG. 19 using different combinations of RNA-FM embeddings according to an embodiment and conventional inputs.

FIGS. 21A and 21B show graphs further illustrating results obtained from the deep learning framework of FIG. 19 using different combinations of RNA-FM embeddings according to an embodiment and conventional inputs.

### DETAILED DESCRIPTION

The following description of exemplary embodiments of the invention is presented for the purpose of illustration and description. It is not intended to be exhaustive or to limit the invention to the precise form described, and persons skilled in the art will appreciate that many modifications and variations are possible. The embodiments have been chosen and described in order to best explain the principles of the invention and its practical applications to thereby enable others skilled in the art to best utilize the invention in various embodiments and with various modifications as are suited to the particular use contemplated.

Existing approaches to predicting RNA structure and/or function rely on annotated data sets, which typically contain a relatively small fraction of the known RNA sequences. For example, while millions of RNA sequences are known, relatively few of them have been annotated. Thus, existing models for deep-learning-based prediction of structure or function for a given RNA sequence depend on data sets of up to about 30,000 sequences. This limits the ability of such models to generalize.

Certain embodiments of the invention provide deep-learning-based models that can exploit the much larger data set of unannotated RNA sequence data, which implicitly contains evolutionary and structural information of RNA sequences. According to some embodiments, an RNA foundation model (also referred to herein as an “RNA-FM”) can be based on a language model (e.g., a transformer model). The RNA foundation model can be initially trained in a self-supervised and task-agnostic manner to generate output embeddings in a high-dimensional space from input RNA sequences. In this training process, the RNA foundation model can learn sequential distributions and patterns that capture aspects of the underlying structural and functional information. Training can be followed by a task-specific stage, in which the pre-trained RNA foundation model can generate sequence embeddings that can be used as inputs to task-specific downstream machine-learning models. If desired, the RNA foundation model can be fine-tuned for a particular downstream task, e.g., by using a lightweight prediction layer. With the powerful representation learned from unannotated RNA data (e.g., ncRNA data), an RNA foundation model can significantly improve performance across a broad range of downstream tasks related to prediction of RNA structure and/or function, with only minor modifications to the architecture of the downstream model. In some embodiments, a trained RNA-FM can be made available as a service. For instance, a server that hosts a pre-trained RNA-FM can receive queries that include RNA sequences from client systems (e.g., systems that perform downstream tasks) and can return responses that include the corresponding RNA-FM embeddings. In some embodiments, a trained RNA-FM can produce interpretable RNA representations, which reflect evolutionary information and can be used to infer evolutionary trends of variants of a virus or other pathogen.

### RNA-FM Based on Language Model

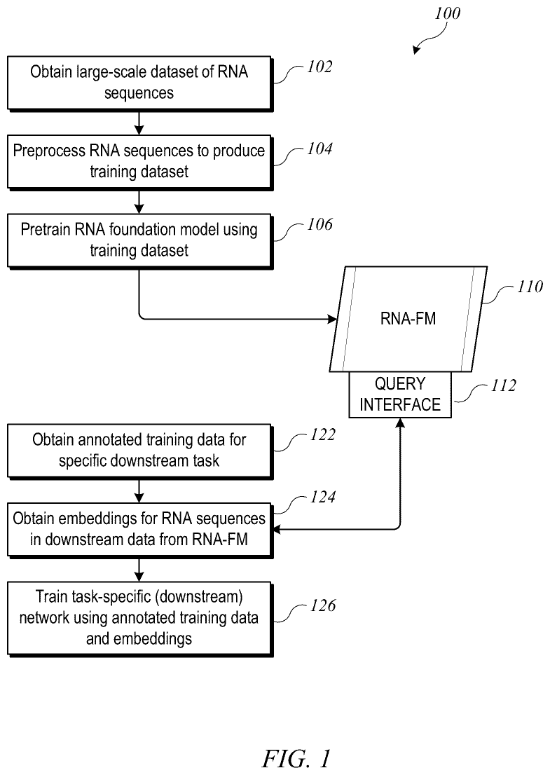

FIG. 1 show a flow diagram of a process 100 according to some embodiments. Process 100 includes two phases. In the first phase, an RNA foundation model (or RNA-FM) 110 of RNA sequences is constructed. RNA-FM 110 can be a machine-learning language model, such as a transformer-based model, that can be trained to receive an RNA sequence as input and to output an embedding of the RNA sequence in a high-dimensional space (referred to herein as an RNA-FM embedding). For instance, each nucleotide token can be embedded into a 640-component feature vector, and the embedding for a sequence of length L can have dimension L×640. In the second phase, information provided by RNA-FM 110 (e.g., an embedding corresponding to a given RNA sequence) is incorporated into a downstream machine-learning process that learns a specific task, such as predicting a structural or functional characteristic of RNA from an input sequence. In some embodiments, process 100 is applied to ncRNA sequences.

Blocks 102-106 relate to training RNA-FM 110. At block 102, a large-scale dataset of RNA sequences is obtained. The dataset can be unannotated (e.g., providing no information other than the nucleotide sequences). One example of a suitable large-scale dataset is the existing “RNAcentral” dataset, which combines ncRNA sequences across 47 different databases, adding up to around 27 million RNA sequences in total.

At block 104, RNA sequences in the large-scale dataset are preprocessed to produce a training dataset for RNA-FM 110. For example, all instances of ‘T’ can be replaced with ‘U’, resulting in a dataset involving 4 main types of bases: ‘A’, ‘C’, ‘G’, ‘U’ (or 16 counted types of combination in total: ‘A’, ‘C’, ‘G’, ‘U’, ‘R’, ‘Y’, ‘K’, ‘M’, ‘S’, ‘W’, ‘B’, ‘D’, ‘H’, ‘V’, ‘N’, ‘-’.) Duplicate sequences can be eliminated, e.g., by defining a cutoff for degree of similarity and removing duplicates if the similarity exceeds the cutoff. In some embodiments, the known cd-hit-est algorithm can be applied with a cut-off at 100% (i.e., only identical sequences are deduplicated). In one example, a training dataset for RNA-FM 110 consists of 23.7 million ncRNA sequences. This data set is sometimes referred to herein as “RNAcentral100.”

At block 106, RNA-FM 110 is trained (also referred to herein as “pre-training” to distinguish from subsequent training of downstream tasks) using the training dataset from block 104. In an example implementation, RNA-FM 110 contains a stack of transformer encoder blocks (e.g., 12 blocks encoder blocks) similar to BERT (Bidirectional Encoder Representations from Transformers, a well-known language model first published in 2018). Each encoder block consists of a feed-forward layer and a multi-head self-attention layer. Layer normalization and residual connections are applied before and after every block, respectively, and the output tensor from each encoder block has the same size as the input. For an RNA sequence with length L, an example implementation of RNA-FM 110 takes raw sequential nucleotide tokens as input, and an input embedding layer maps each nucleotide token into a 640-dimensional vector, thus resulting in an L×640 embedding matrix. The embedding matrix then proceeds through each encoder block to produce an output embedding. Training can use self-supervised training as in BERT. For example around 15% of nucleotide tokens can be randomly replaced with a special mask token. If the i-th token is chosen, then the i-th token is replaced with: (1) the [MASK] token 80% of the time; (2) a random token 10% of the time; or (3) the unchanged i-th token 10% of the time. The model can then be trained with masked language modelling (MLM) by predicting tokens that were replaced by the [MASK] token (e.g., using a Softmax layer applied to the output embedding) and applying cross-entropy loss. This training strategy can be formulated as an objective function as follows:

$${ℒ}_{ℳℒℳ} = {E}_{x ∼ X} {E}_{{x}_{ℳ} ∼ x} {\sum }_{i ∈ ℳ} - log p ( {x}_{i} \vert {x}_{/ ℳ} ) . \qquad ( 1 )$$

In Eq. (1), a set of indices [symbol] are randomly sampled from each of the input sequences x (15% among the whole sequence) and masked by replacing the true token at each index i with some other mask token. Next, for each masked token, when the masked sequence ( [symbol] ) is given as context, the objective function of Eq. (1) will minimize the negative log-likelihood of the corresponding true nucleotide xi. This objective function can capture dependencies between the masked portion and the remaining parts of the input sequence, which enables accurate predictions for masked positions. Thus, training RNA-FM 110 via Eq. (1) drives the network to gain a deep understanding and rich representation of each sequential token.

Once trained, RNA-FM 110 can provide a query interface 112 that can receive a query RNA sequence and respond by providing a corresponding output embedding based on the trained RNA-FM. Query interface 112 can communicate between processes running on the same computer system or between different computer systems via a network connection. It should be noted that RNA-FM 110 is based on sequences of nucleotide tokens and is agnostic to structural or functional characteristics of the RNA. As such, RNA-FM 110 can be trained once, after which the output embeddings it produces can be used by multiple different downstream processes to perform different tasks related to predicting structural and/or functional characteristics of RNA based on an input RNA sequence.

For instance, at block 122 of process 100, annotated training data for a specific downstream task can be obtained. The downstream task can be any computer-based process, particularly a machine-learning process, that predicts structural or functional characteristics of RNA (e.g., ncRNA) from an input RNA sequence. Examples of structure-related tasks include predicting secondary structure, 3D closeness and/or 3D distance, and so on. Examples of function-related tasks include predicting untranslated region (UTR) function, RNA-protein interactions, and so on. The annotated training data can include RNA sequences and annotations providing information about the characteristic that the task should learn to predict. It should be understood that the training data used for the downstream task can but need not overlap with the data set used to train RNA-FM 110.

At block 124, embeddings can be obtained for the RNA sequences in the annotated training data by querying RNA-FM 110 (via query interface 112). At block 126, the downstream task can be trained using the annotated training data. More specifically, the downstream task can be trained to take as input an RNA sequence and a corresponding output embedding from RNA-FM 110 and predict a particular structural or functional characteristic of the RNA sequence. Annotated training data can be used in a supervised learning process to train the downstream task. Any number of different downstream tasks can be trained in a similar manner, without further training of RNA-FM 110. Specific examples are described below.

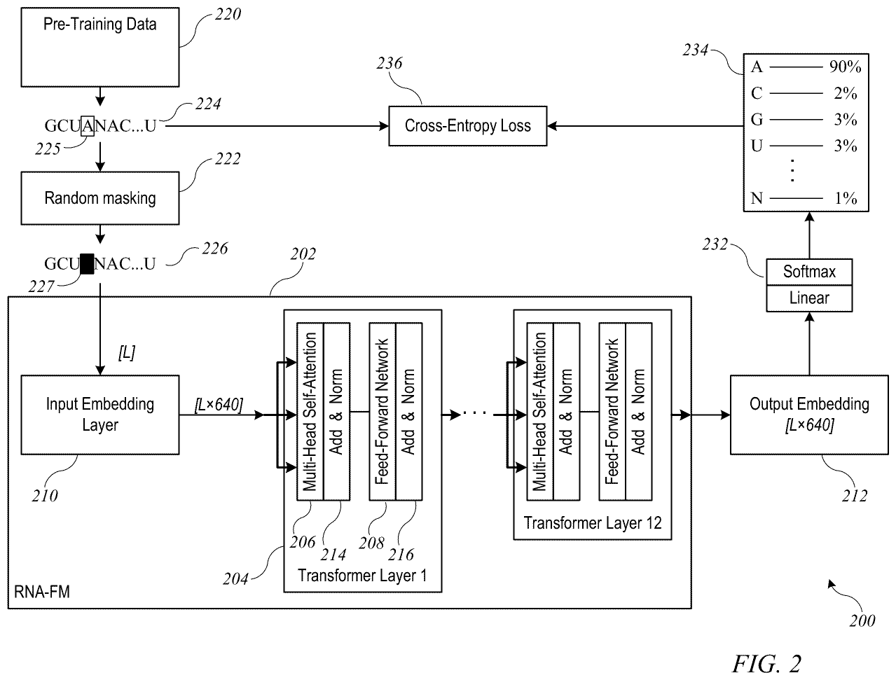

By way of illustration, specific examples of an RNA foundation model and downstream tasks will now be described. FIG. 2 shows an overview of a system 200 for training an RNA foundation model according to some embodiments. System 200 includes RNA-FM 202, which can be an implementation of RNA-FM 110. RNA-FM 202 can incorporate multiple transformer layers 204; in some embodiments, there are 12 transformer layers 204. Each transformer layer 204 includes a 20 multi-head self-attention layer 206 and a 640 hidden size feed-forward layer 208. Layer normalization and residual connections are applied before and after every layer, as indicated by blocks 214, 216. RNA-FM 202 also includes an input embedding layer 210 that receives an RNA sequence, which can be in a standardized form (e.g., replacing all ‘T’ with ‘U’ as described above), and maps each nucleotide token in the RNA sequence into a 640-dimensional vector, thus resulting in an L×640 input embedding matrix. The transformer layers 204 preserve the size of the embedding matrix. Thus, the output embedding 212 can be an L×640 embedding matrix.

RNA-FM 202 can be trained using a large training data set 220, such as the RNAcentral100 dataset described above, which contains over 23 million sequences representing more than 800,000 species. To implement self-supervised learning, input sequences 224 in the training data set 220 are subject to a random masking module 222 that randomly masks a certain fraction (e.g., 15%) of tokens, as described above. For example, given an input sequence 224 from training data set 220, the nucleotide 225 in the fourth position may be replaced with a mask token 227 as shown in masked sequence 226. The training task for RNA-FM 202 is to learn to reconstruct the masked nucleotides. For this purpose, softmax layer 232 can be applied to output embedding 212 to generate predictions 234 of the likelihood that the nucleotide 225 masked by mask token 227 is a particular type. Based on the predictions and the original input sequence, cross-entropy loss module 236 can compute cross-entropy loss as described above, and the cross-entropy loss can be used to modify weights in transformer layers 204 and input embedding layer 210.

RNA-FM 202 is illustrative and that variations and modifications are possible The size of the embedding, the number of transformer layers, training data, and training processes and parameters can be all be varied. As noted, training of an RNA-FM can be based on just the RNA sequence data, with no annotations as to structure, function or source of a particular sequence. The transformer model, however, can extract latent information about structure, function, and/or source and reflect such information in the output embeddings.

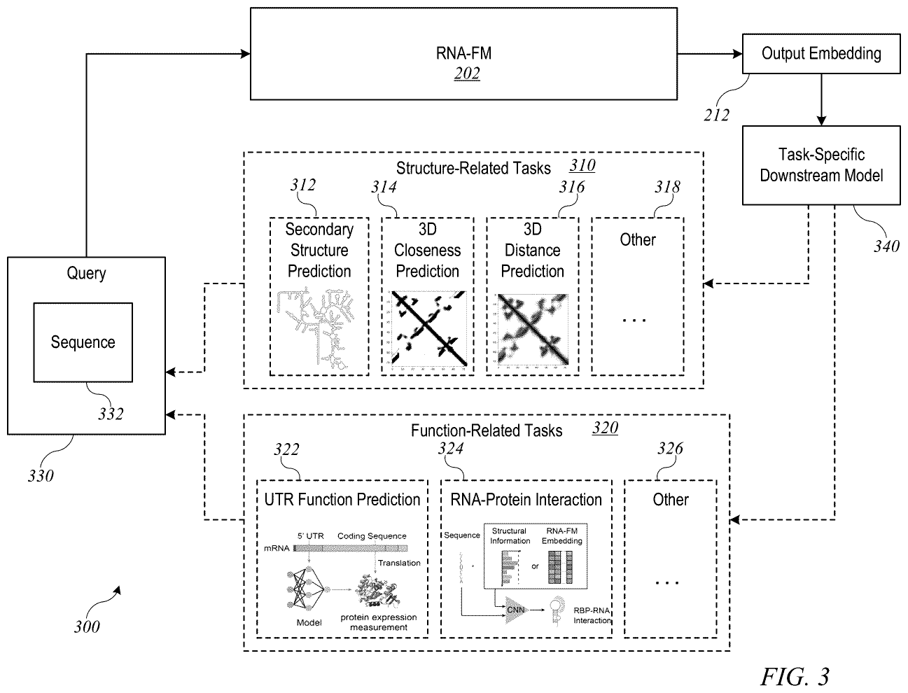

FIG. 3 shows an overview of a system 300 for downstream use of an RNA foundation model (e.g., RNA-FM 202 of FIG. 2) according to some embodiments, including fine-tuning and application to specific tasks related to prediction of RNA structure and function. It is assumed that RNA-FM 202 has been trained as described above. System 300 can use RNA-FM 202 to obtain embeddings (also referred to as features) to be used as input features for one or more specific structure-related tasks 310 and/or one or more function-related tasks 320. Each downstream task can be implemented using a machine-learning model (which can be any type of machine learning model) that is trained for that specific task. Any number and combination of downstream tasks can be supported. Examples of structure-related tasks 310 include secondary structure prediction task 312, 3D closeness prediction task 314, and 3D distance prediction task 316. Specific implementations and considerations for these tasks are described below. Other structure-related task(s) 318 may also be supported. Examples of function-related tasks 320 include UTR function prediction task 322 and RNA-protein interaction prediction task 324. Specific implementations and considerations for these tasks are described below. Other function-related task(s) 326 may also be supported.

For a given task, a query 330 can that includes an RNA sequence 332 can be presented to RNA-FM 202. As shown, RNA sequence 332 can be extracted from query 330 and processed through (pre-trained) RNA-FM 202 to produce an output embedding 212, also referred to herein as an “RNA-FM embedding.” For an input RNA sequence 332 of length L, the output embedding 212 can be an L×640 embedding matrix. Output embeddings corresponding to queried sequences 332 can be provided to one or more task-specific downstream models 340. Different task-specific downstream models 340 can be trained for specific structure-related tasks 310 and/or function-related tasks 320; specific examples of task-specific downstream models 340 for various tasks are described below. Supervised, unsupervised, and/or semi-supervised learning can be used to train a given instance of task-specific downstream model 340.

Integration of (pre-trained) RNA-FM 202 into downstream applications can include one or both of two schemes: feature-based training and fine-tuning. For the feature-based scheme, the network parameters of RNA-FM 202 are frozen and fed to downstream models. For some fine-tuning schemes, further training of RNA-FM 202 can take place together with training of downstream modules. For other fine-tuning schemes, the downstream model can incorporate one or more transformer layers or other layers that operate on the RNA-FM embeddings to produce fine-tuned embeddings for a particular downstream task.

### EXAMPLES

Example applications of RNA-FM 202 will now be described. For purposes of illustration, an embodiment of RNA-FM 202 was implemented as described above with reference to FIG. 2 and was trained on the RNAcentral100 data set described above, using a computer system with eight A100 GPUs with 80 GB memories for a period of one month. An inverse square root learning rate schedule was adopted with a 0.00011 base learning weight, 0.01 weight decay, and 10,000 warm-up steps. Maximum length of input sequences was set to 1024, to reduce memory consumption and increase batch size, thereby speeding up training. This particular implementation is referred to herein as “the exemplary RNA-FM”; however, it should be understood that other training paradigms and computer hardware can be used and that “exemplary” means “illustrative.”

To illustrate the ability of RNA-FM embeddings to encode structural, functional, and evolutionary properties, RNA-FM embeddings were used without downstream processing to create an “RNA atlas” to organize RNAs according to functional, structural, and/or evolutionary relationships. In addition, to illustrate the ability of RNA-FM embeddings to enhance downstream analysis tasks related to prediction of RNA structure, function, or evolution, To further illustrate uses of RNA-FM embeddings, a number of different downstream tasks were implemented. For each downstream task, a task-specific machine-learning model was implemented that could receive RNA-FM embeddings as input features, either alone or in combination with other input features such as raw sequence data or other available data. Each model was trained using annotated data sets, then tested. To illustrate the effect of RNA-FM embeddings, corresponding models receiving only conventional input features were also trained and tested, and results were compared. The following sections describe specific examples.

### Example 1: RNA Atlas

RNA functions and structures vary across different RNA types, and embodiments of an RNA-FM are capable of encoding these rich properties within the output embeddings. To illustrate the encoding of functional and structural properties, the exemplary RNA-FM was applied to generate embeddings for the known RNA universe, including housekeeping RNA (rRNA, tRNA) and regulatory RNA (lncRNA, snoRNA, miRNA, siRNA, snRNA, piRNA), as well as other types. An RNA Atlas was generated by subsampling the RNAcentral100 data set with a maximum of 10,000 samples per each RNA type. Each instance from different RNA families was represented by a 640-dimensional vector by averaging across its RNA-FM embedding (which has dimensions L×640 for a sequence of length L) at each position in the sequence. UMAP (described in McInnes, L., Healy, J. & Melville, J. “UMAP: Uniform manifold approximation and projection for dimension reduction,” arXiv preprint arXiv:1802.03426 (2018)) was applied to reduce the 640-dimensional vectors into two dimensions (2D), thereby projecting the vectors onto a plane. For comparison, embeddings were also generated using a random-initialized (untrained) version of the exemplary RNA-FM (referred to herein as a “Random RNA-FM” model), and a one-hot encoding of raw sequence data was also generated; projections onto a plane were generated in a similar manner for all three data sets.

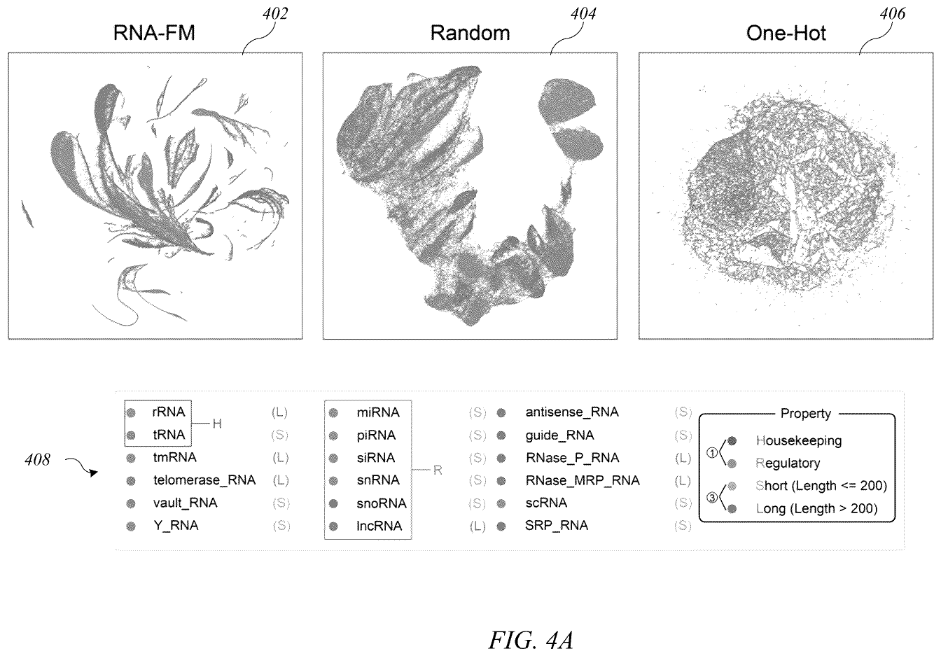

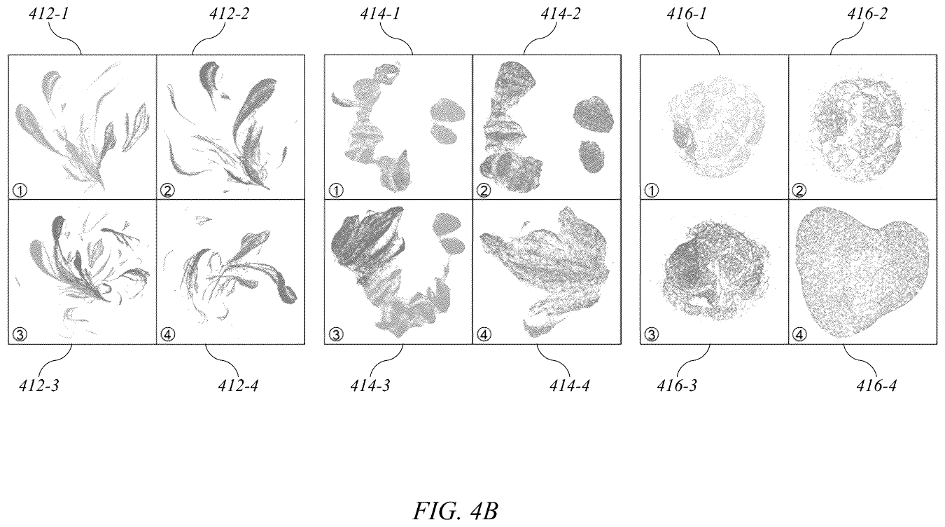

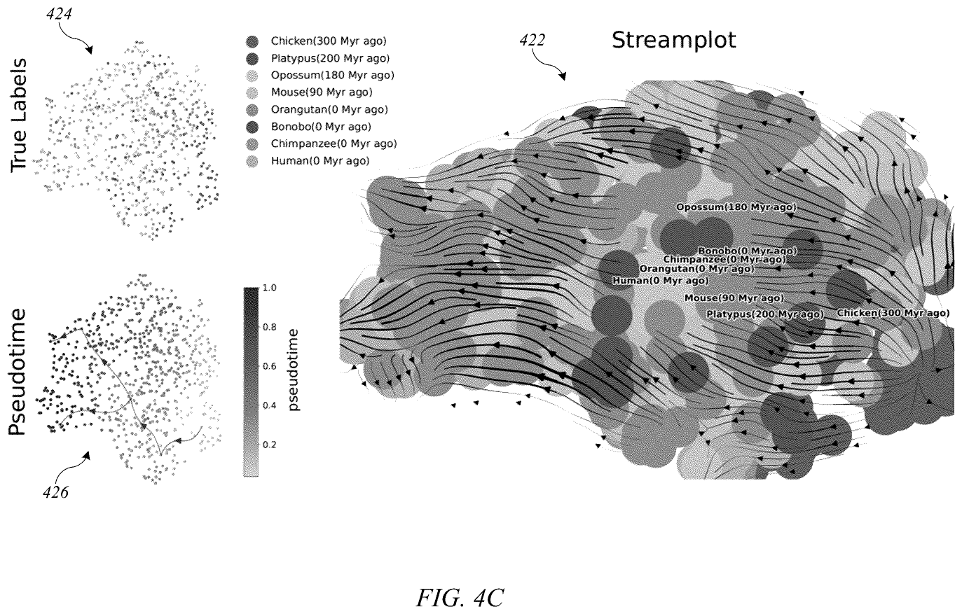

FIGS. 4A-4C show graphs of results, which illustrate that RNA-FM embeddings can be understood as encoding functional, structural, and evolutionary properties of RNA sequences. FIG. 4A shows UMAP projections of different ncRNA types into a 2D space. Plot 402 shows the UMAP projection obtained from output embeddings of the exemplary RNA-FM. Plot 404 shows the corresponding projection for output embeddings of the Random RNA-FM, and plot 406 shows projections obtained using one-hot encoding of raw sequence data. A legend applicable to FIGS. 4A and 4B is shown at 408. Plot 402 shows that output embeddings from the exemplary RNA-FM are organized by structure and function properties, with clear boundaries between clusters. In contrast, plot 404 shows that the Random RNA-FM provides only vague clustering structure, and plot 406 shows that one-hot encoding provides no apparent structure information. This example illustrates that an RNA-FM can learn structural or functional information beyond the primary structure, such that instances with similar properties are grouped in the output embedding space.

FIG. 4B shows UMAP projections, separated according to various categories. For this purpose, four categories are defined: (1) housekeeping ncRNAs; (2) regulatory ncRNAs; (3) long ncRNAs (more than 200 nucleotides); and (4) short ncRNAs (up to 200 nucleotides). Plots 412-1 through 412-4 show projections of output embeddings for the exemplary RNA-FM for each of the four categories 1-4. Plots 414-1 through 414-4 show corresponding projections of output embeddings for the Random RNA-FM, and plots 416-1 through 416-4 show corresponding projections for the one-hot encoding. Plots 412-1 and 412-3 suggest that the exemplary RNA-FM discriminates housekeeping and regulatory categories more effectively than short and long categories, which suggests that the RNA-FM encoding emphasizes structural or functional similarity more than length. This is desirable since RNAs with different length could share the same functions while RNAs of similar length could differ significantly in function. A closer examination of plots 412-2 and 412-4 likewise suggests that RNA embeddings produced by the exemplary RNA-FM aggregate or separate according to the similarity of structures and functions.

To explore evolutionary information contained in RNA-FM embeddings, trajectory inference (or pseudotemporal ordering) techniques that are commonly applied in single-cell transcriptomics were applied to a selected subset of lncRNAs. RNAs in the selected subset can be classified according to different types of species in which they occur. To model their evolutionary relationship, the exemplary RNA-FM was used to generate embeddings for the selected subset, and trajectory inference was carried out using VIA (described in Stassen S. V. et al., “Generalized and scalable trajectory inference in single-cell omics data with VIA,” Nature Communications 12, 1-18 (2021)). FIG. 4C shows the resulting streamplot 422, as well as projections for the true labels (scatter plot 424) and pseudotime (scatter plot 426). While the exemplary RNA-FM did not reliably distinguish the RNAs according to species, streamplot 422 suggests that RNA-FM embeddings can provide a roughly accurate evolutionary trend of different species according to the ground-truth timeline. It should be noted that this result was achieved without incorporating evolutionary or species features into the training data, which used only the pure RNA sequences. This example suggests that RNA-FM implementations can extract implicit genetic messages and encode the output embeddings with evolutionary information.

### Example 2: RNA Structure Prediction

RNA structure often determines its function, and therefore structural understanding is important for many RNA-related applications. However, structure of ncRNAs has been experimentally determined for only a tiny fraction (less than 0.001%) of known ncRNAs, due to the high cost of suitable experiments and the structural instability of RNA. Accordingly, computational approaches to predicting 3D structure from an RNA nucleotide sequence are of considerable interest. To illustrate the performance of RNA-FM based approaches, the following downstream tasks were studied: (1) secondary structure prediction; (2) contact map prediction; and (3) distance map prediction. For simplicity of implementation, a simple 2D ResNet was adopted as a unified downstream prediction module for all three structure prediction tasks rather than designing a separate framework for each task. The 2D ResNet was implemented as a deep residual network consisting of 32 blocks, where each block contained two convolution layers with a filter size of 64. Input to the 2D ResNet was the outer concatenation of output embeddings obtained for the query sequences using the exemplary RNA-FM.

RNA secondary structure prediction. RNA secondary structure reflects hydrogen bonds in the primary sequence and can be rapidly formed from the primary sequence by pairing bases with hydrogen bonds. Secondary structure is generally more stable and more accessible in cells than tertiary forms, making the ability to predict secondary structure important for higher-order structure predictions and even function predictions.

To illustrate the effect of RNA-FM embeddings on secondary structure prediction capability, the following benchmark data sets were considered: (1) RNAStralign (described in Tan, Z. et al., “Turbofold II: RNA structural alignment and secondary structure prediction informed by multiple homologs,” Nucleic Acids Research 45, 11570-11518 (2017)), which consists of 37,149 structures from eight RNA types; (2) ArchiveII (described in Sloma, M. F. & Matthews, D. H., “Exact calculation of loop formation probability identifies folding motifs in RNA secondary structures,” RNA 22, 1808-1818 (2016)), which consists of 3,975 RNA structures from ten RNA types; and (3) bpRNA-1m (described in Singh, J. et al., “RNA secondary structure prediction using an ensemble of two-dimensional deep neural networks and transfer learning,” Nature Communications 10, 1-13 (2019)). Each data set was preprocessed by removing similar sequences (with 80% sequence identity cutoff) and restricting maximum sequence length to below 500 nucleotides. The preprocessed data set containing 13,419 sequences was randomly split into 10,814 RNAs for training (TRO), 1300 for validation (VL0), and 1,305 for testing (TS0). The models were evaluated using ArchiveII600 (a subset with lengths less than 600) and TS0.

For comparison, twelve conventional approaches to secondary structure prediction were also tested, including: UFold (described in Fu, L., et al., “UFold: Fast and accurate RNA secondary structure prediction with deep learning,” bioRxiv 202-08 (2021)); E2Efold (described in Chen, X., et al., “RNA secondary structure prediction by learning unrolled algorithms,” arXiv preprint arXiv:2002.05810 (2020)); LinearFold (described in Huang, L., et al., “LinearFold: Linear-time approximate RNA folding by 5′-to-3′ dynamic programming and beam search,” Bioinformatics 35, i295-i304 (2019)); Mfold (described in Zuker M., “Mfold web server for nucleic acid folding and hybridization prediction,” Nucleic Acids Research 31, 3406-3415 (2003)); RNAstructure (described in Reuter, J. S. & Mathews, D. H., “RNAstructure: Software for RNA secondary structure prediction and analysis,” BMC Bioinformatics 11, 1-9 (2010)); RNAfold (described in Sato, K., et al., “RNA secondary structure prediction using deep learning with thermodynamic integration,” Nature Communications 12, 1-9 (2021)); CONTRAfold (described in Do, C. B., et al., “CONTRAfold: RNA secondary structure prediction without physics-based models,” Bioinformatics 22, e90-e98 (2006)); SPOT-RNA (described in Singh, J., et al., “RNA secondary structure prediction using an ensemble of two-dimensional deep neural networks and transfer learning,” Nature Communications 10, 1-13 (2019)); RNAsoft (described in Andronescu, M., et al., “RNAsoft: A suite of RNA secondary structure prediction and design software tools,” Nucleic Acids Research 31, 3416-3422 (2003)); MXfold2 (described in Singh, J., et al., “RNA secondary structure prediction using an ensemble of two-dimensional deep neural networks and transfer learning,” Nature Communications 10, 1-13 (2019)); Contextfold (described in Zakov, S., et al., “Rich parameterization improves RNA structure prediction,” Journal of Computational Biology 18, 1525-1542 (2011)); and Eternafold (described in Wayment-Steele, H. K., et al., “RNA secondary structure packages ranked and improved by high-throughput experiments,” BioRxiv (2020)).

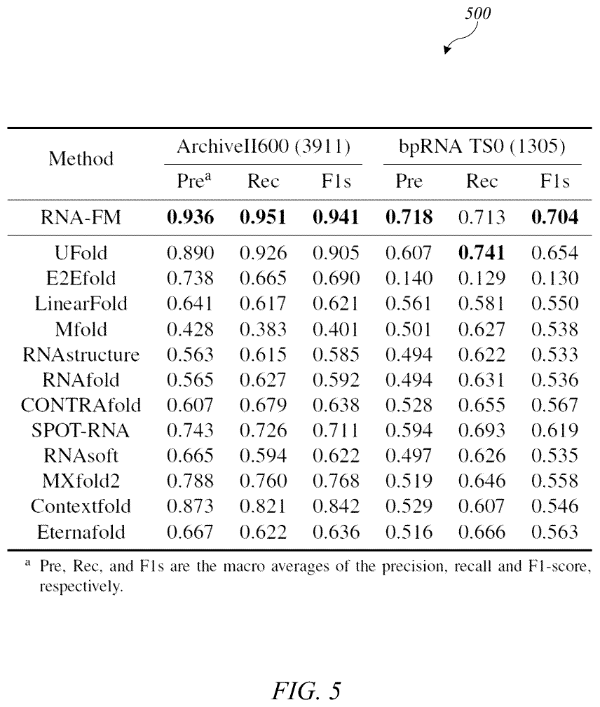

FIG. 5 shows a table 500 summarizing results of this study. Metrics of precision (Pre), recall (Rec), and F1-score (F1s) are shown for the ArchiveII data set and for the bpRNA TS0 data set. As shown, the downstream ResNet using RNA-FM embeddings as inputs outperformed all other approaches with regard to almost all metrics.

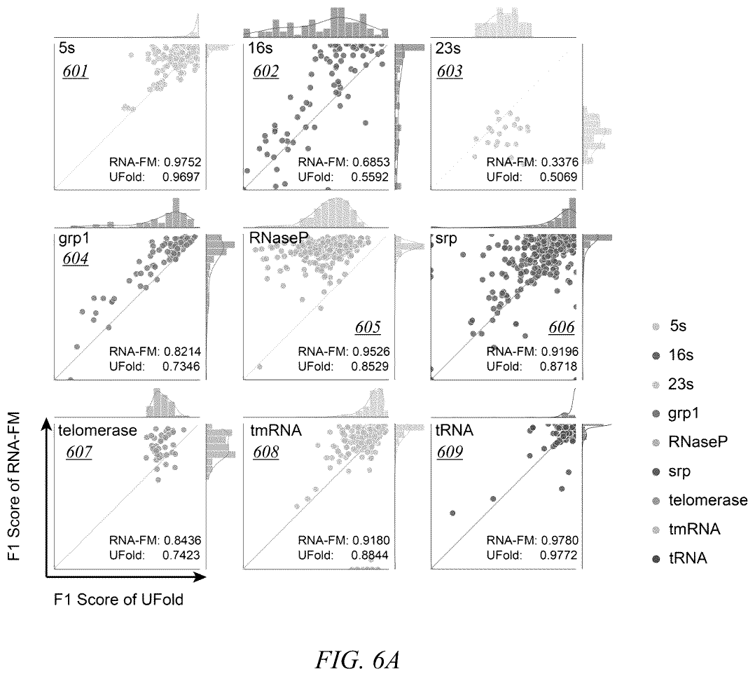

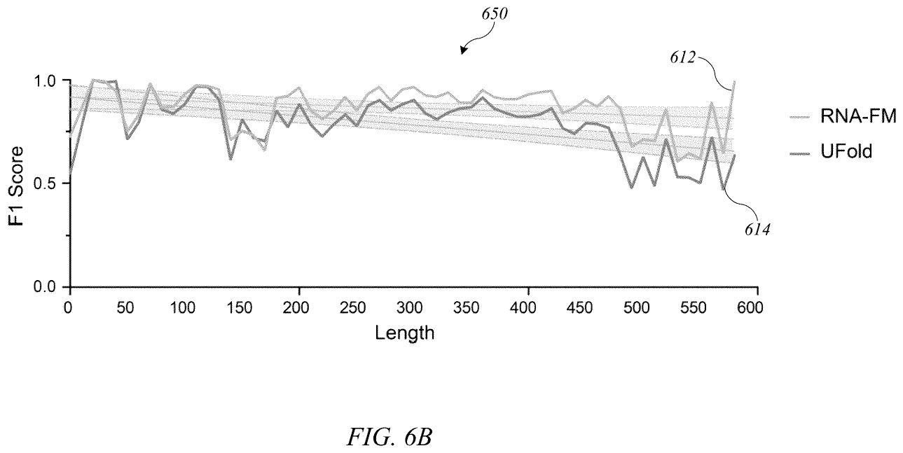

In addition, a head-to-head comparison of the RNA-FM and UFold approaches was performed using the ArchiveII600 data set. FIGS. 6A and 6B show graphs comparing the performance of the RNA-FM and UFold approaches. FIG. 6A shows scatter plots 601-609 comparing the F1 scores across nine RNA types, with performance of the RNA-FM approach on the y-axis and performance of the UFold approach on the x-axis. Each point represents an RNA structure. Almost all points are above the diagonal, indicating that the RNA-FM approach outperforms the UFold approach in almost all instances. FIG. 6B shows a graph 650 of F1 scores as a function of RNA sequence lengths for the RNA-FM (line 652) and UFold (line 654) approaches. The RNA-FM approach consistently outperforms the UFold approach, particularly for sequences with length over 150.

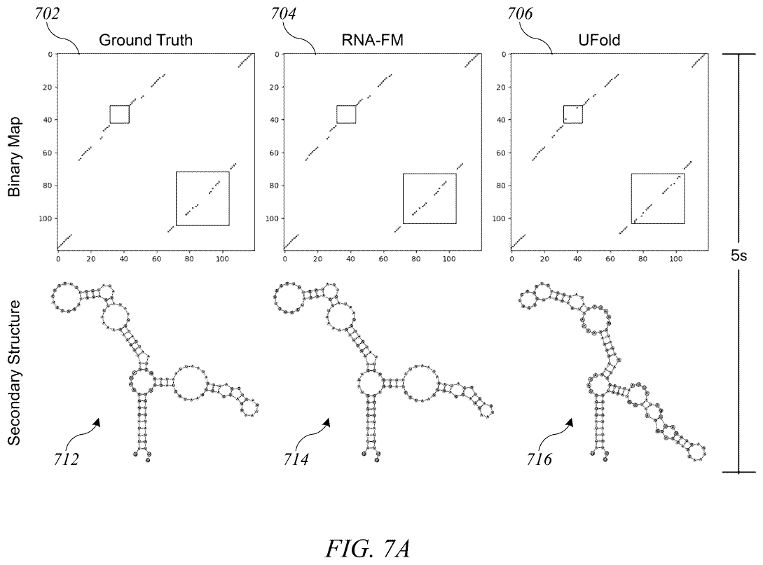

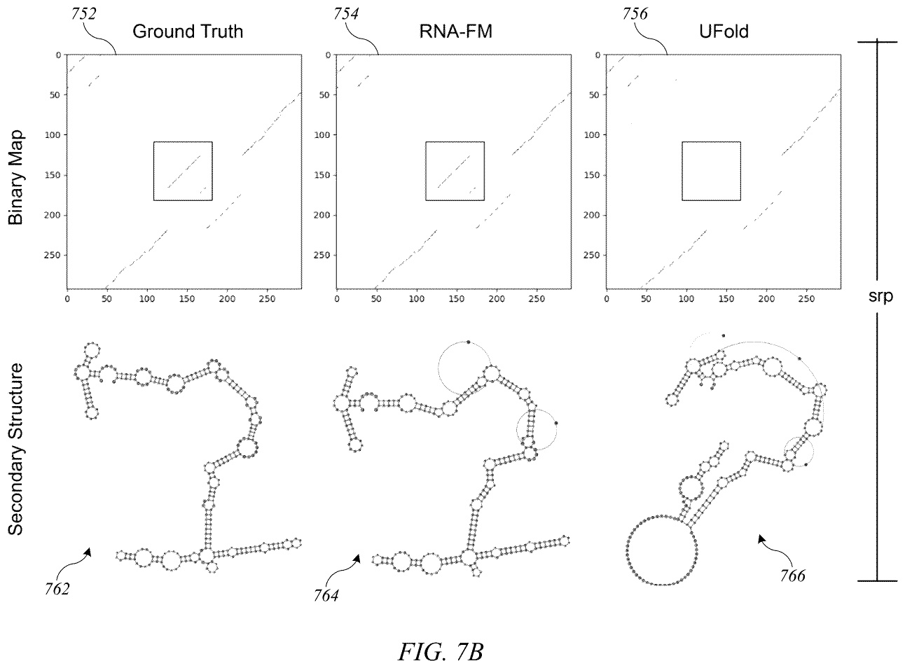

FIGS. 7A and 7B show binary probability maps predicted by the model with a threshold of 0.5 and visualizations of the secondary structure predictions of two randomly selected example sequences. FIG. 7A shows a first example, and FIG. 7B shows a second example. Binary probability maps (702, 704, 706 in FIG. 7A; 752, 754, 756 in FIG. 7B) reflect the probability of hydrogen bonds between particular positions in the sequence for ground truth (maps 702, 752), predictions from the RNA-FM approach (maps 704, 754), and predictions from the UFold approach (maps 706, 756). Secondary structure visualizations (712, 714, 716 in FIG. 7A; 762, 764, 766 in FIG. 7B) are derived from the corresponding probability maps and reflect ground truth (visualizations 712, 762), predictions from the RNA-FM approach (visualizations 714, 764) and predictions from the UFold approach (visualizations 716, 766). As can be seen in FIGS. 7A and 7B, the probability maps from the RNA-FM approach (second column) are more robust, less noisy, and much closer to the ground truth (first column) than are corresponding probability maps from the UFold approach. Similarly, as can be seen from the visualizations, the RNA-FM approach also generates secondary structures more similar to the ground truth than UFold does.

These results obtain despite the fact that UFold utilizes prior knowledge to model the probability of pairing while the RNA-FM approach uses only the RNA sequence data and RNA-FM embeddings as input, with structure information being learned in the ResNet. Thus, this example illustrates the power of the RNA-FM approach.

RNA 3D closeness prediction. Although secondary structure can reveal parts of the relationship between pairs of bases in RNA, it is usually treated as a prior result that provides a constraint in subsequent structure modeling. To obtain more precise structures, various tasks have been proposed to generate more strict constraints for subsequent structure modeling. RNA 3D closeness is one such task. RNA 3D closeness indicates that two arbitrary bases have a tertiary interaction if their distance is under a certain threshold. This concept originates from the “contact” concept in the protein field. RNA 3D closeness uses a 2D matrix to represent pairwise tertiary inter-nucleotide interactions (rather than the 2D flat relationship in secondary structure). The distance is defined as the minimal atomic distance between arbitrary pairs of bases, and for purposes of illustration, a minimum distance is set at 8 Å.

To illustrate the effectiveness of RNA-FM embeddings for a 3D structure prediction task, the benchmark data sets used by RNAcontact (described in Sun, S., et al., “RNA inter-nucleotide 3D closeness prediction by deep residual neural networks,” Bioinformatics 37, 1093-1098 (2021)) were selected. The RNAcontact data set is based on a set of non-redundant RNA 3D structures containing 1786 entries with initial resolution less than 4 Å. Following conventional preprocessing, sequences with length less than 32 nucleotides or greater than 1000 nucleotides were removed, as were sequences with redundancy over 80% or with too few (fewer than 5) positive points. Of the remaining sequences, 221 were used for training (denoted as TR221) and 80 were used for testing (denoted as TE80). Ground truth was computed from the Protein Data Bank (PDB) files following steps outlined above. Other features involved in the RNAcontact pipeline include covariance of multiple sequence alignment (MSA) and secondary structure predicted by the PETfold based on MSA.

The following approaches to 3D closeness prediction were tested: (1) RNAcontact with an ensemble of 100 models (denoted Seq); (2) a ResNet32 model with MSA covariance features (denoted Cov); (3) a ResNet32 model with MSA covariance and secondary structure features (denoted Cov+SS); (4) a ResNet32 model with RNA-FM embeddings from the exemplary RNA-FM as inputs (denoted RNA-FM); and (5) a ResNet32 model with RNA-FM embeddings from the exemplary RNA-FM as inputs and transfer learning (denoted RNA-FM(TL)).

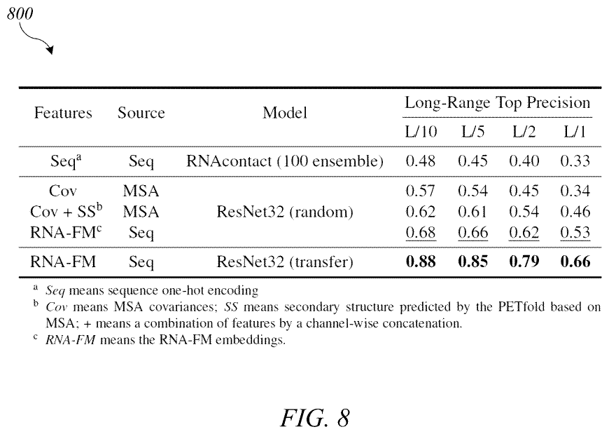

FIG. 8 shows a table 800 of long-rang top precisions of the different models on the TE80. The first row (Seq) shows results from RNAcontact with the sequence encoding as input; an ensemble result over 100 models is shown. The next four rows show results predicted by ResNet32 models with different input features (approaches 2-5 listed above). RNA-FM (fourth row) outperforms other models by a significant margin, and performance can be further improved with the application of transfer learning (fifth row).

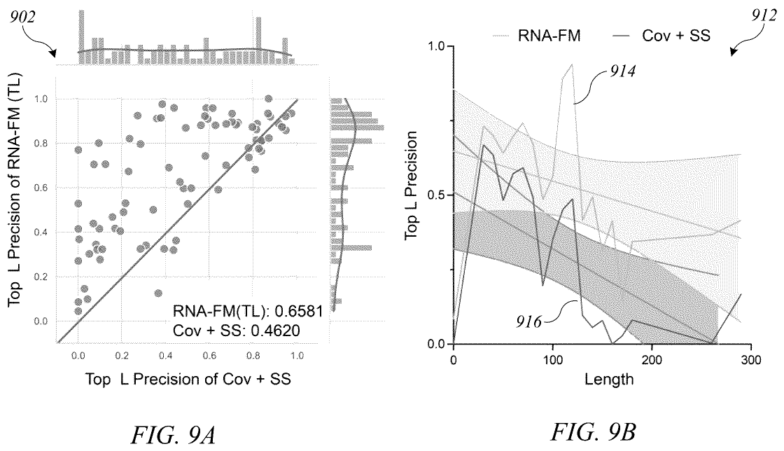

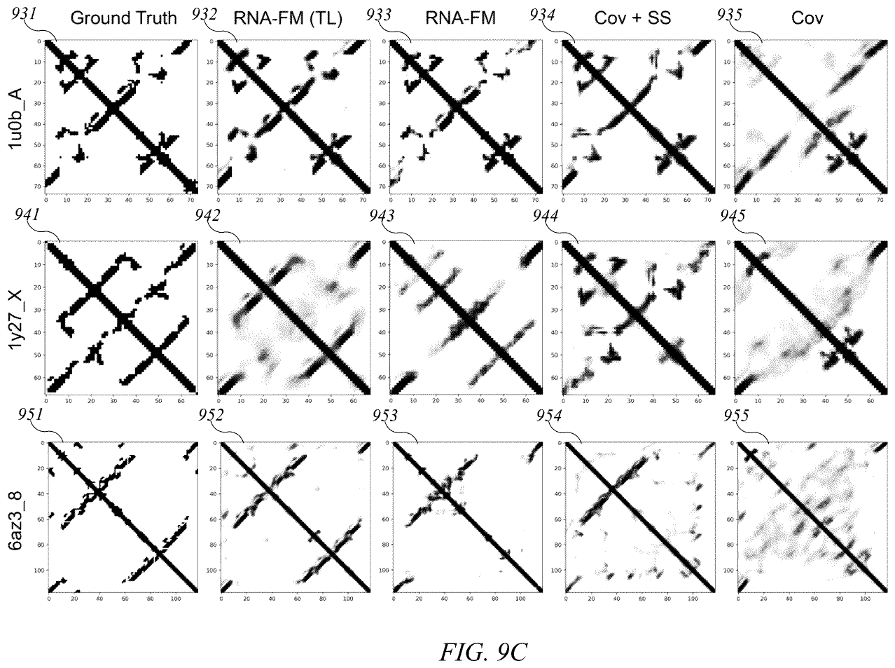

Further illustrating the results, FIGS. 9A-9C show graphical comparisons of the performance of different approaches. FIG. 9A shows a scatter plot 902 of long-range Top-L precision distribution across all samples in TE80. The y-axis of scatter plot 902 represents RNA-FM(TL), and the x-axis represents the Cov+SS approach. RNA-FM(TL) matches or exceeds the Cov+SS approach on 77.5% of instances of all RNA types, as indicated by most points being above the diagonal. FIG. 9B shows a graph 912 of Top-L precision as a function of the input RNA sequence length for the RNA-FM(TL) approach (line 914) and the Cov+SS approach (line 916). RNA-FM(TL) outperforms Cov+SS across all sequence lengths. FIG. 9C presents predicted probability maps for three randomly selected example RNA sequences in TS0 using different approaches. Maps 931-935 (top row), maps 941-945 (middle row), and maps 951-955 (bottom row) correspond to the three different example RNA sequences. For each example, maps are generated using each of the based approaches described above: maps 931, 941, 951 represent ground truth (to which other maps can be compared); maps 932, 942, 952 were generated using RNA-FM(TL) approach; maps 933, 943, 953 were generated using RNA-FM approach (with random learning); maps 934, 944, 954 were generated using Cov+SS approach, and maps 935, 945, 955 were generated using Cov approach. With standalone RNA-FM embedding as input, the downstream model generated visualizations (maps 933, 943, 953) much closer to ground truth (maps 931, 933, 935) than models using Cov+SS (maps 934, 944, 954) or Cov (maps 935, 945, 955). Further, as illustrated in maps 932, 942, 952 RNA-FM(TL) performs even better than other approaches. It should also be noted that unlike other features generated from MSA data, RNA-FM embeddings are obtained from pure sequences, eliminating the time consuming step of doing multiple sequence alignment.

RNA 3D distance map. An RNA 3D distance map defines distances between arbitrary bases in the primary sequence. Such a map provides more information than RNA secondary structure prediction and 3D closeness prediction. However, predicting structure in this manner is currently an underdeveloped task for RNA.

Using the exemplary RNA-FM, a task was defined to construct a distance prediction. The RNAcontact data set described above was used. Ground truth distance maps for RNA sequences were generated from their PDB files based on the minimal atomic distance between arbitrary bases. A set of 1036 sequences were used for training and another 100 were used for validation and testing. Distance was limited to the range from 0 to 20 Å, with all distances over 20 Å set equal to 20 Å; distances were then normalized to a range [0, 1] by dividing by 20. An end-to-end differentiable model for 3D structure prediction was defined. The model incorporated 4 Evoformers as its backbone, with an equivalent graph transformer (EGNN) stacked on top as a 3D atom coordinate predictor. Root mean square deviation (RMSD) in distance was used as the objective for training. Using each of six different combinations of inputs, instances of the 3D structure prediction model were separately optimized for more than 10,000 steps. The six combinations of inputs were: (1) Sequences using a one-hot encoding (Seq); (2) sequences plus secondary structures predicted using E2Efold (SS+Seq); (3) SS+Seq, further augmented by covariances derived from MSA (SS+Cov+Seq); (4) RNA-FM embeddings from the exemplary RNA-FM; (5) RNA-FM embeddings augmented with Seq (RNA-FM+Seq); and (6) RNA-FM embeddings augmented with Seq and MSA covariances (RNA-FM+Cov+Seq).

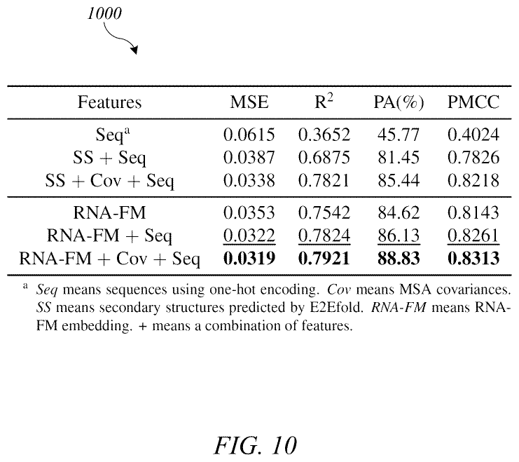

FIG. 10 shows a table 1000 summarizing 3D distance prediction performance of models with the six different inputs on TE80. Metrics considered include mean square error (MSE), R², pixel accuracy (PA), and product moment correlation coefficient (PMCC). As shown, RNA-FM (fourth row) significantly outperformed Seq (first row). Further, augmenting RNA-FM embeddings with the one-hot sequence encoding improved over SS+Cov+Seq. This example suggests that RNA-FM embeddings provide the most explicitly helpful information for the distance prediction task, while eliminating the time-consuming MSA searching step required to generate covariances. Further performance advantages were obtained when RNA-FM was combined with sequences and covariances.

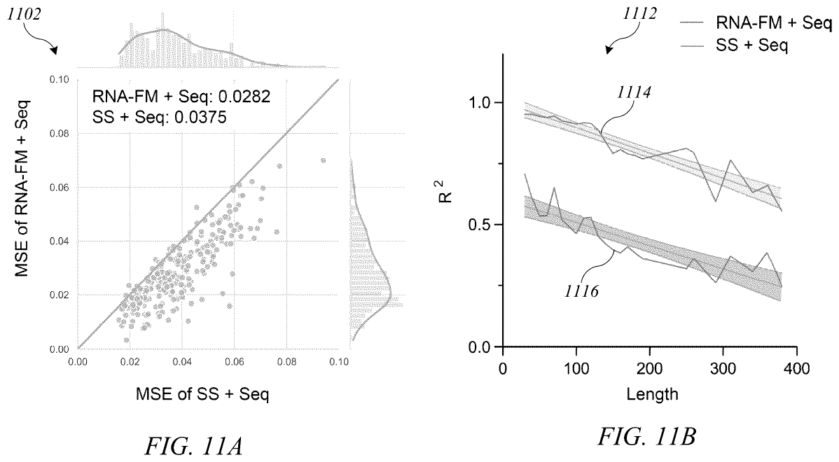

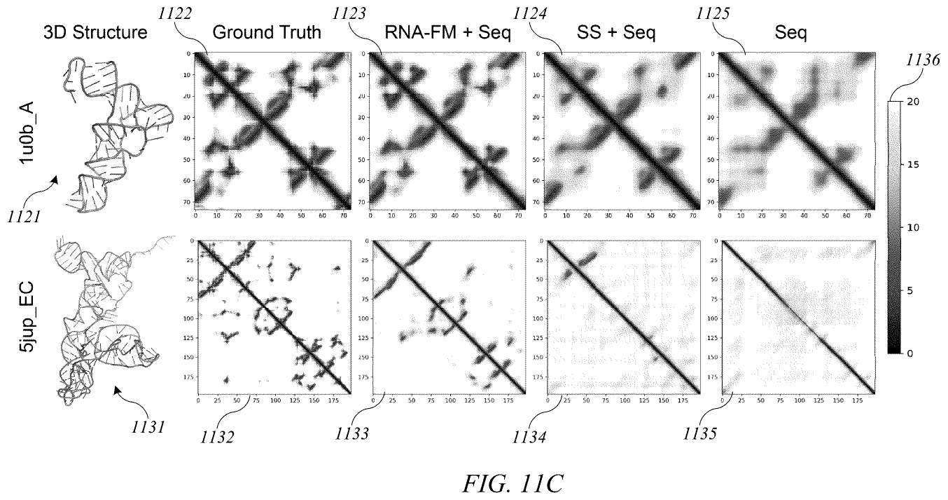

Further illustrating the comparison, FIGS. 11A-11C show graphical illustrations comparing results from 3D structure models with different inputs. FIG. 11A shows a scatter plot 1102 of MSE; the x-axis corresponds to SS+Seq, and the y-axis corresponds to RNA-FM+Seq. Each point represents the MSE for a different RNA structure in the testing data set. Almost all points are below the diagonal, which indicates that RNA-FM yields more accurate results in nearly all instances.

FIG. 11B shows a graph 1112 of R² as a function of sequence length for RNA-FM+Seq (line 1114) and for SS+Seq (line 1116). RNA-FM+Seq outperforms SS+Seq for all sequence lengths.

FIG. 11C shows probability maps for two different testing samples. Ground truth 3D structures 1121, 1131 are shown in the first column, and the corresponding ground truth probability maps 1122, 1132 are shown in the second column. Probability maps 1123, 1133 generated using RNA-FM+Seq are shown in the third column; probability maps 1124, 1134 generated using SS+Seq are shown in the fourth column; and probability maps 1125, 1135 generated using Seq are shown in the fifth column. Probability maps 1122-1125 and 1132-1135 are color-coded to represent probable distances according to legend 1136 at the right. As can be seen, RNA-FM+Seq is closer to ground truth than other inputs. Standalone sequence data (Seq) performs worst, only capturing distance values along a diagonal.

RNA 3D reconstruction. The ultimate goal of RNA structure prediction is to enable reconstruction of the 3D shape of RNA. In an experiment, output embeddings from the exemplary RNA-FM were combined with existing optimization tools to obtain 3D approximations of the shape. Specifically, secondary structures were generated based on RNA-FM embeddings, as described above. For comparison with the state of the art, secondary structures were also generated using UFold. The 3D structures were optimized using existing RNA 3D modeling tools, including 3dRNA and FARFAR2 (described in Watkins, A. M., et al., “FARFAR2: Improved de novo Rosetta prediction of complex global RNA folds,” Structure 28, 963-976 (2020)).

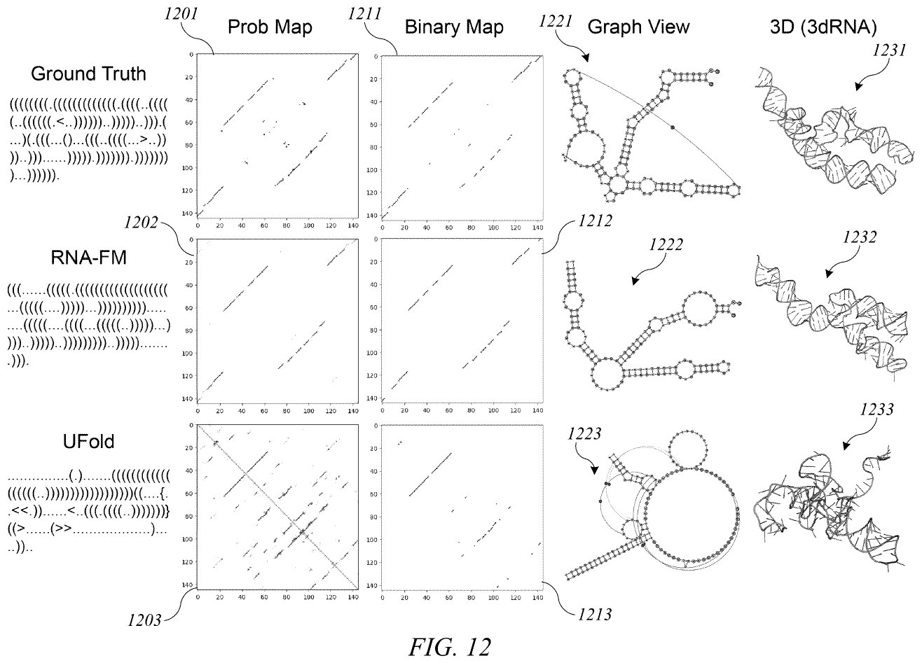

An example of reconstructing a 3D structure is shown in FIG. 12, for an instance 5m73-1-A from the PDB test set. Specifically, FIG. 12 shows probability maps 1201-1203, binary maps 1211-1213, graph views 1221-1223, and 3D renderings 1231-1233, generated using three different approaches. As shown in the first row, for ground truth (extracted from PDB), a probability map 1201 was used to generate a binary map 1211, from which a graph view 1221 and a 3D model 1231 were produced. Similarly, as shown in the second row, a probability map 1202 generated using RNA-FM embeddings (as described above) was used to generate a binary map 1212 from which a graph view 1222 and a 3D model 1232 were produced. For comparison, as shown in the third row, a probability map 1203 generated using UFold was used to generate a binary map 1213, from which a graph view 1223 and a 3D model 1233 were produced. RNA-FM produced a root mean square deviation (RMSD) of around 7.91, significantly better than the result from UFold, which had RMSD of 25.70. It is noted that the ground truth secondary structure produced RMSD of 13.96 (higher than for RNA-FM), which suggests that error may be introduced in the 3D structure modeling process.

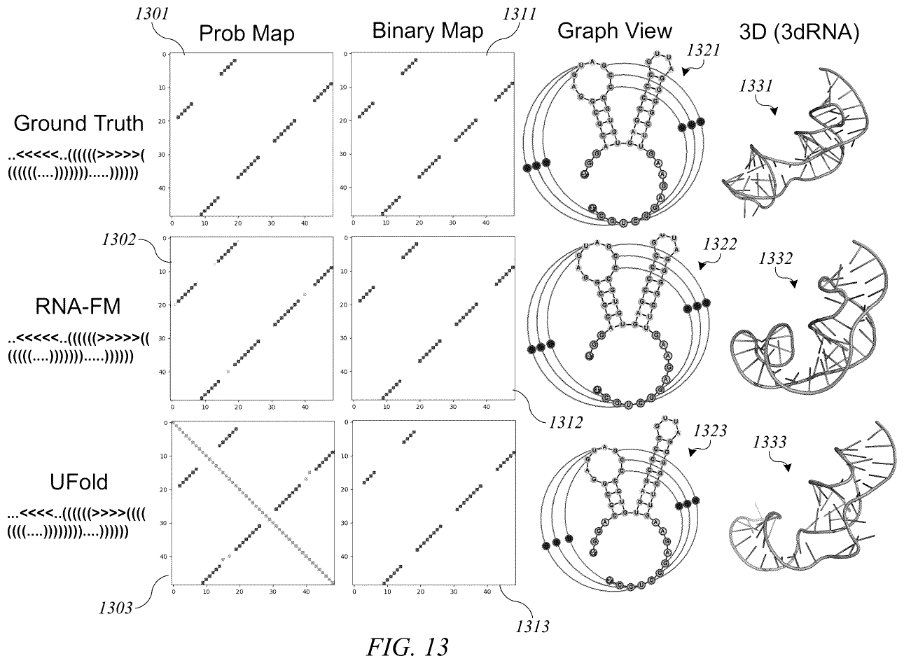

Another example of reconstructing a 3D structure is shown in FIG. 13 for the DCS-PK element in the 5′ UTR flanking region of the Zika virus (ZIKV). The DCS-PK is a pseudoknot found in the coding region that helps enhance genome cyclization during replication. Similarly to FIG. 12, FIG. 13 shows probability maps 1301-1303, binary maps 1311-1313, graph views 1321-1323, and 3D renderings 1331-1333, generated using three different approaches. As shown in the first row, for ground truth (extracted from PDB), a probability map 1301 was used to generate a binary map 1311, from which a graph view 1321 and a 3D model 1331 were produced. Similarly, as shown in the second row, a probability map 1302 generated using RNA-FM embeddings (as described above) was used to generate a binary map 1312 from which a graph view 1322 and a 3D model 1332 were produced. For comparison, as shown in the third row, a probability map 1303 generated using UFold was used to generate a binary map 1313 from which a graph view 1323 and a 3D model 1333 were produced.

As these example shows, the RNA-FM embeddings enable the downstream models to capture specific details of distance data that are not available using only sequences and secondary-structure data. Accordingly the secondary structure predictions and resulting 3D structures can be more precise where RNA-FM embeddings are used as input features.

### Example 3: SARS-CoV-2 Secondary Structure and Evolution

Many communicable illnesses are caused by viruses. Spread of such viruses can have significant public health consequences, including mass death and/or illness, potentially with lasting effects. Understanding of the genome structure and evolution of viruses can enable development of effective vaccines and antiviral treatments and is therefore an important tool for preventing the spread of viral disease.

To illustrate the power of RNA-FM for understanding of virus genome structure and evolution, downstream tasks involving analysis of genome structure and evolution were performed on strains of Severe acute respiratory syndrome coronavirus 2 (SARS-CoV-2), the causal pathogen of the COVID-19 pandemic.

In a first downstream task, RNA-FM embeddings from the exemplary RNA-FM were used to predict secondary structures of certain key regulatory segments of the SARS-CoV-2 reference genome (Refseq accession number NC_045512.2). For this analysis, 3′UTR, 5′UTR, and other segments were sampled from the entire genome with length 29870. Secondary structure prediction was performed using a model as described above.

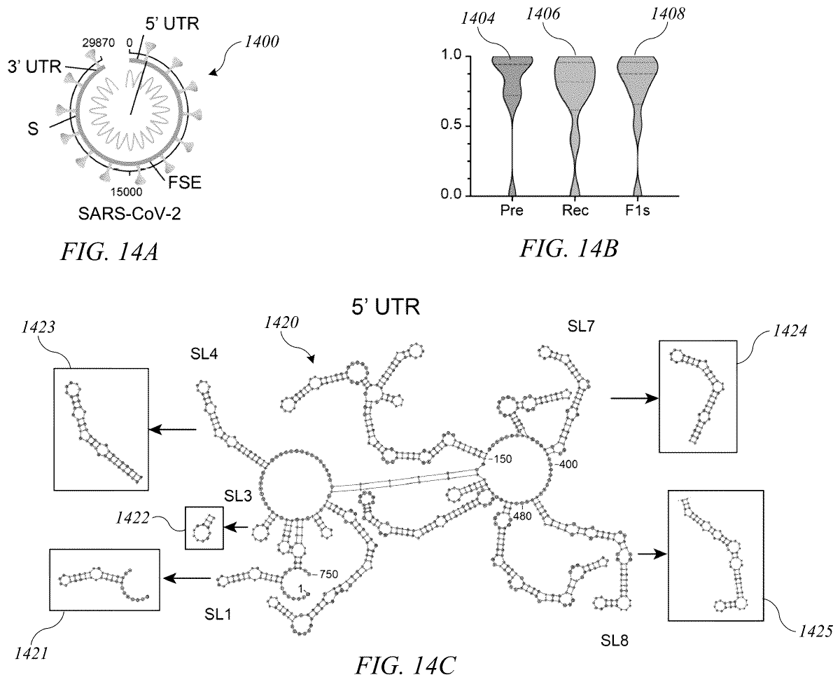

FIG. 14A shows a schematic diagram 1400 representing SARS-CoV-2 and indicating the sampled segments. FIG. 14B shows violin plots of secondary structure prediction performance parameters: precision (Pre) 1404, recall (Rec) 1406, and F1 score (F1s) 1408 for the sampled segments. FIG. 14C shows a visualization of RNA secondary structure predictions in the 5′UTR region. Visualization 1420 represents ground truth for the 5′UTR region. Boxes 1421-1425 show secondary structure predictions from RNA-FM for fragments within the 5′UTR region. As can be seen, the predictions in boxes 1421-1425 are a close match to ground truth visualization 1420.

In a second downstream, evolutionary trajectory of variants of SARS-CoV-2 was explored by applying RNA-FM to the whole genome. Although the exemplary RNA-FM was not pre-trained for whole-genome modeling, it was assumed that aggregation of RNA-FM embeddings for fragments of the whole genome would still characterize the genome sufficiently to enable study of the evolution of the genome. It was also noted that the length of the SARS-CoV-2 genome (around 30,000 nucleotides) is far longer than the maximum sequence length (1024 nucleotides) of the exemplary RNA-FM. Accordingly, for feature extraction, a fixed-length window of 1022 nucleotides on non-overlapping subsections of the whole virus genome was employed to extract the RNA-FM embeddings. Embeddings were aggregated by averaging, and the final standard length vector was used as the RNA-FM embedding for the whole genome. For each variant, a maximum of 100 instances were sampled from all sequences available at SARS-CoV-2 Data Hub. Trajectory inference was carried out using VIA (as in Example 1 described above), with k in the k-nearest neighbors algorithm set to 120 and the Alpha type set as the root.

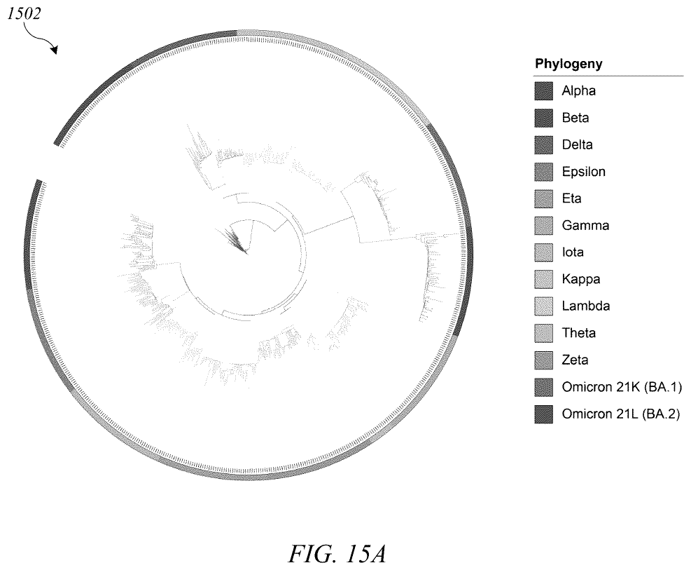

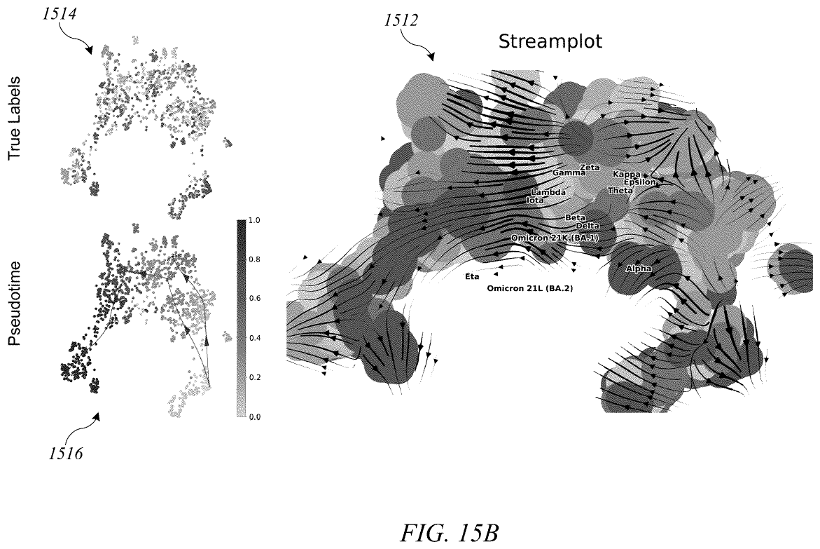

FIG. 15A shows a phylogenetic tree 1502 generated using FastME (described in Desper, R. & Gascuel, O., “Fast and accurate phylogeny reconstruction algorithms based on the minimum evolution principle,” in International Workshop on Algorithms in Bioinformatics, 375-374 (Springer, 2002)), which is treated as ground truth for the evolutionary trend of COVID-19, from Alpha type to Omicron variants (March 2020 through April 2022). FIG. 15B shows the resulting streamplot 1512, as well as projections for the true labels (scatter plot 1514) and pseudotime (scatter plot 1516), of an evolutionary trajectory inferred using RNA-FM genome-level embeddings. The results are highly consistent with the ground truth. In particular, the predicted evolution begins with the Alpha type and ends with the Omicron variant by April 2022, including evolution from Omicron 21K to Omicron 21L.

This example shows that regulatory elements of the virus genome can be important for understanding virus evolution. This example also shows the capability of RNA-FM embeddings to reveal core structure messages and evolutionary trend information of a virus such as SARS-CoV-2 and its variants. Thus, RNA-FM embeddings of the kind described herein may facilitate research into viral diseases such as COVID-19.

### Example 4: RNA-Protein Interaction Modeling

Protein-RNA interactions play important roles in a variety of activities, including cell-signaling, post-transcriptional regulation and protein synthesis. Accordingly, the ability to predict RNA binding proteins corresponding to particular RNAs is of interest, and some previous work has been performed. For instance, PrismNet (described in Sun, L., et al., “Predicting dynamic cellular protein-RNA interactions by deep learning using in vivo RNA structures,” Cell Research 31, 495-516 (2021)) uses deep learning to integrate experimentally measured in vivo RNA secondary structures with information about RNA binding protein sites in the same cell lines to predict RNA-protein interactions within a given cell line.

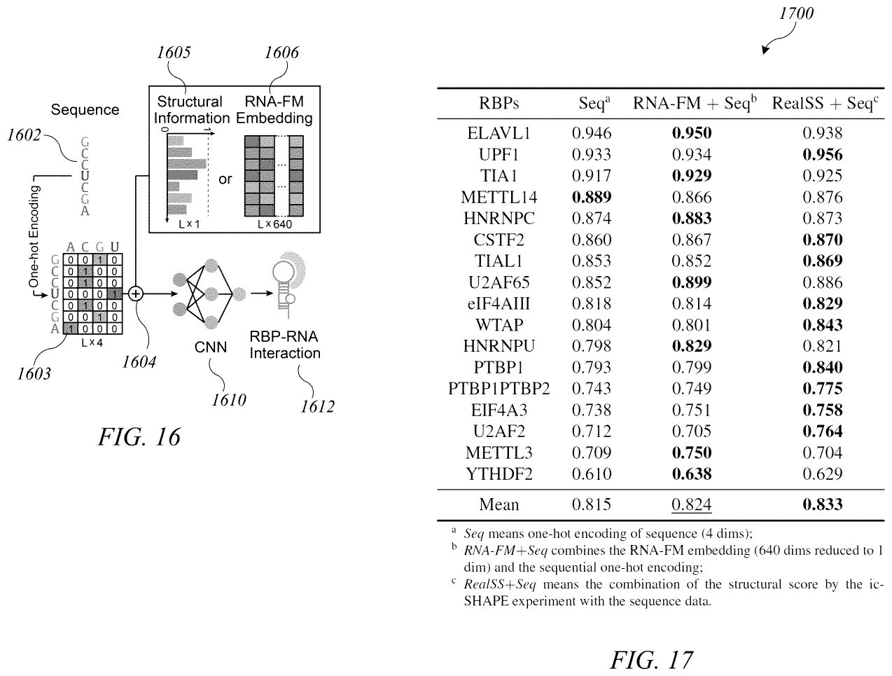

To illustrate the effectiveness of RNA-FM embeddings for an RNA-protein interaction task, various implementations of PrismNet were compared. The HeLa cell line was used as the data set, divided into 17 different subsets each having corresponding RNA binding proteins (RBPs). To predict whether an input RNA can bind with proteins in each subset, PrismNet was implemented using each of the following as inputs: (1) raw sequences (Seq); (2) Seq combined with in vivo secondary structures (RealSS) as previously determined using in vivo click selective 2′-hydroxyl acylation and profiling experiment (icSHAPE) (described in Spitale, R. C., et al., “Structural imprints in vivo decode RNA regulatory mechanisms,” Nature 519, 486-490 (2015)); and (3) Seq combined with embeddings generated by the exemplary RNA-FM. In each case, the same PrismNet architecture was used. FIG. 16 shows a schematic illustration of the PrismNet implementations. Sequence data 1602 was encoded with a one-hot encoding 1603. For different implementations, the one-hot encoding 1603 was augmented (operation 1604) with either in vivo secondary structural information 1605 or the RNA-FM embedding 1606 of the sequence (or neither). The resulting feature vector was input into the PrismNet convolutional neural network 1610 to produce predictions 1612 of RBP-RNA interaction with each of the 17 subsets.

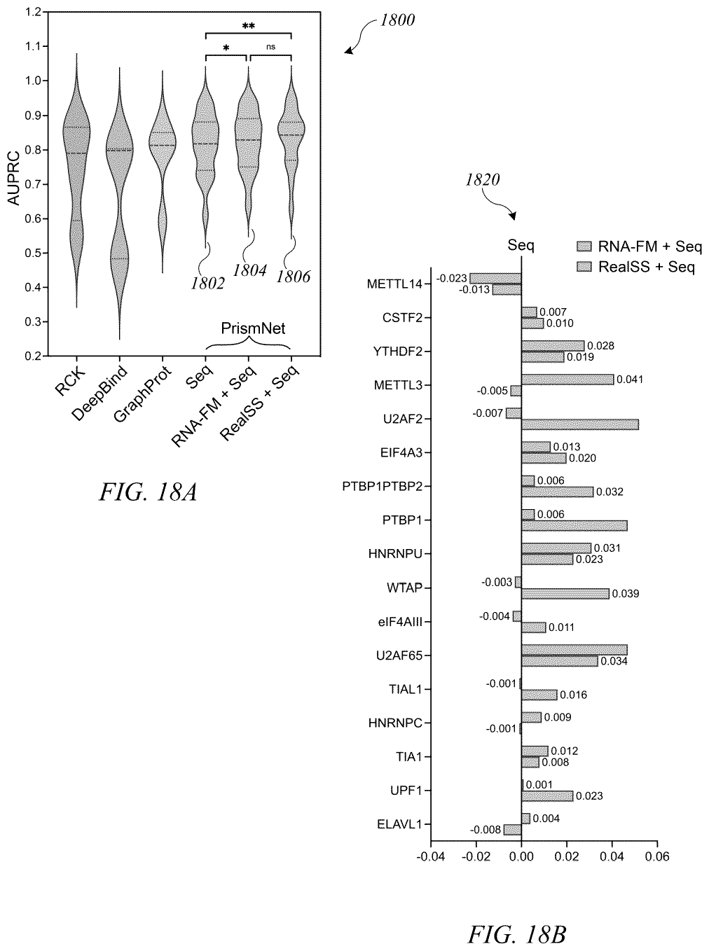

Outputs from the different PrismNet implementations were compared, to determine whether using RNA-FM embeddings as input could match the performance of the in vivo secondary structures. FIG. 17 shows a table 1700 summarizing the results for different RBPs and different input data sets using area under the precision recall curve (AUPRC) as a figure of merit. Further illustrating these results, FIG. 18A shows violin plots 1800 of the AUPRCs for PrismNet implementations with different input feature sets, including Seq (plot 1802), RNA-FM+Seq (plot 1804), and RealSS+Seq (plot 1806). Also shown for comparison are results from three conventional methods: RCK (described in Orenstein, Y., et al., “RCK: Accurate and efficient inference of sequence- and structure-based protein-RNA binding models from RNAcomplete data,” Bioinformatics 32, i351-i359 (2016); DeepBind (described in Alipanahi, B., et al., “Predicting the sequence specificities of DNA- and RNA-binding proteins by deep learning,” Nature Biotechnology 33, 831-839 (2015)); and GraphProt (described in Maticzka, D., et al., “GraphProt: Modeling binding preferences of RNA-binding proteins,” Genome Biology 15, 1-18 (2014)). FIG. 18B shows a histogram plot 1820 of AUPRCs for different RBPs using the PrismNet implementations. For each RBP, the Seq result was taken as the baseline, and the height and direction of each bar represents the difference between the RNA-FM+Seq result (or the RealSS+Seq result) and the Seq result. As FIGS. 17 and 18A-18B show, RNA-FM+Seq and RealSS+Seq both outperformed Seq on almost all RBPs. While RealSS+Seq has a somewhat higher mean, it should be noted that RNA-FM+Seq outperforms RealSS+Seq on nearly half of the RBPs, which demonstrates that the RNA-FM embeddings can implicitly learn sufficient information about secondary structures from the raw sequences to benefit downstream processes that predict RNA function.

### Example 5: Predicting Regulatory Function of mRNA Untranslated Regions

Modeling of gene expression regulation is of interest in understanding RNA function, and like all functions, gene expression regulation depends on structure. Accordingly, the ability to predict gene expression regulation is of considerable interest.

To illustrate the effectiveness of RNA-FM embeddings for a gene expression prediction task, a downstream task of predicting mean ribosomal load (MRL) based on the 5′ untranslated region (UTR) of messenger RNA (mRNA) was defined. Mean ribosomal load (MRL) reflects how an untranslated region of messenger RNA regulates the expression level of the target protein, and experimental measurements of MRL corresponding to particular UTRs have been obtained using existing techniques such as massively parallel reporter assays and polysome profiling methods. Although the exemplary RNA-FM was trained using only ncRNAs and 5′UTR is part of an mRNA, 5′UTR is a non-coding sequence. Accordingly, it was hypothesized that use of RNA-FM embeddings could facilitate modeling of the relationship between UTR and target protein expression, without additional training of the RNA-FM.

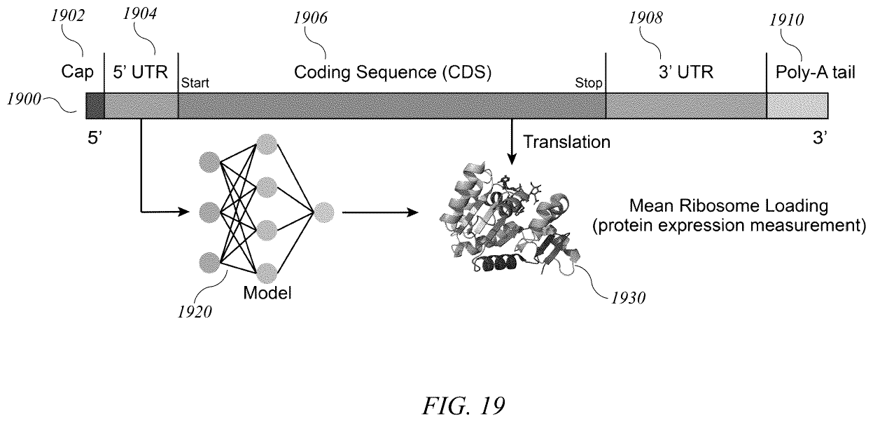

FIG. 19 shows a schematic illustration of a deep learning framework for predicting MRL that was used as the downstream task. An mRMA 1900 includes a cap region 1902, a 5′UTR region 1904, a coding region 1906 (that begins with a start codon and ends with a stop codon), a 3′UTR region 1908, and a poly-A tail 1910. Sequence data for the 5′UTR region 1904 is used as input to a deep learning model 1920 that generates a prediction of MRL. Within a biological cell, coding region 1906 is translated to produce proteins 1930; the amount of proteins 1930 depends at least in part on a regulatory effect of the 5′UTR region. Deep learning model 1920 can be trained to predict the MRL based on the sequence data for the 5′UTR region using supervised learning and experimentally observed MRL. For purposes of illustration, deep learning model 1920 was implemented using a model referred to herein as the “Sample model” (described in Sample, P. J., et al., “Human 5′ UTR design and variant effect prediction from a massively parallel translation assay,” Nature Biotechnology 37, 803-809 (2019)). This model performs a grid search whose framework is constructed as three 1D convolutional layers with 120 filters and a ReLU activation for each layer. The third layer outputs one channel with L length features, which in turn is fed into two fully connected layers to provide a final prediction at a single output node. In the original implementation of the Sample model, inputs to the model were simply the one-hot encodings of the raw sequences (Seq).

Data for this study was obtained from a large-scale synthetic Human 5′UTR library (described in Sample, P. J., et al., “Human 5′ UTR design and variant effect prediction from a massively parallel translation assay,” Nature Biotechnology 37, 803-809 (2019)). The dataset included 83,919 5′UTRs of 75 different lengths and their corresponding MRLs. A validation set was obtained by sampling 7600 sequences equally at each length, and the remainder was used for training. An additional validation set consisting of 7600 real human 5′UTRs with the same length distribution as provided by the library was used for validation to measure the generalization of models.

Five implementations of the Sample model were trained, using five different inputs: (1) one-hot encoded raw sequences (Seq) with dimension L×4; (2) raw sequences plus secondary structure (Seq+SS), formatted as an embedding with dimension L×16; (3) output embeddings with dimension L×640 from the exemplary RNA-FM (RNA-FM); (4) an embedding extracted from a 3D structure prediction framework (EDS); (5) a combination of Seq+SS+RNA-FM; and (6) a combination of Seq+SS+3DS+RNA-FM. In each instance, a linear projection was applied to reduce the embedding dimension to 4, to match the dimension of the one-hot encoding used in the original Sample model.

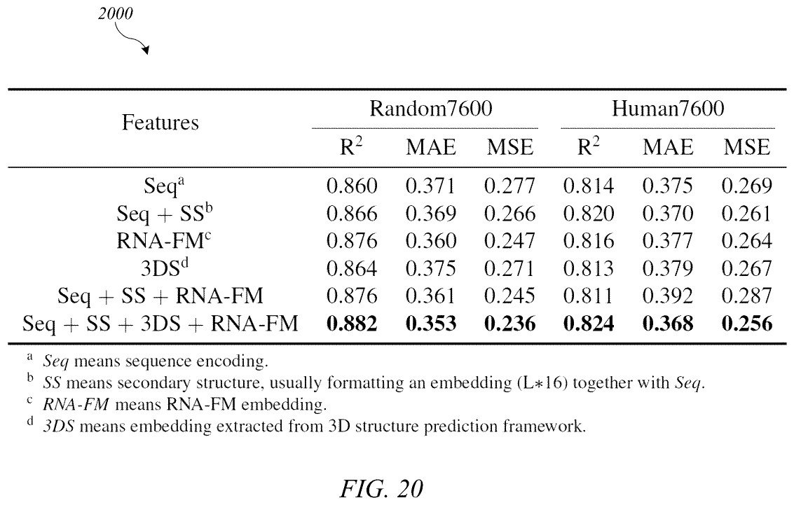

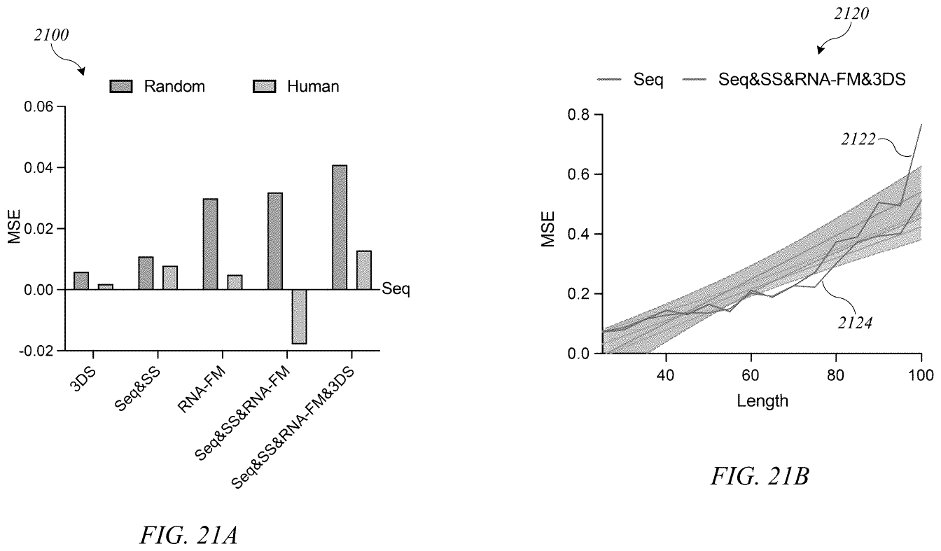

FIG. 20 shows a table 2000 summarizing the results of MRL predictions on the Random7600 (synthetic) and Human7600 (real) data sets. Metrics of R², mean absolute error (MAE), and mean square error (MSE) are presented. FIGS. 21A and 21B show graphs further illustrating the results. FIG. 21A shows a histogram plot 2100 of MSE for different inputs on the random and human validation sets. Seq is used as a baseline, and results for other inputs are shown relative to Seq. FIG. 21B shows a graph 2120 of MSE as a function of sequence length for inputs of Seq (line 2122) and Seq+SS+RNA-FM+3DS (line 2124).

As FIGS. 20 and 21A-21B show, performance of all models on the Human7600 data set was lower than on the Random7600 data set, likely due to differences in data distribution between the real and synthetic data. For either data set, the model with RNA-FM embeddings performed better than the model with raw sequence data (Seq). Incorporating additional structure information (SS and/or 3DS) provides further improvement, and models incorporating RNA-FM embeddings outperform models that do not (Seq and Seq+SS). Like other examples herein, this example illustrates advantages of RNA-FM in biological modeling, even in cases where the system being modeled is not purely related to non-coding RNAs.

### Additional Embodiments

While the invention has been described with reference to specific embodiments, those skilled in the art will appreciate that numerous modifications are possible, including modifications to network structure, loss functions (or objective functions), and other training parameters. Techniques described herein can be applied in a variety of contexts. For instance, different downstream models can be used in combination with output embeddings from a single pre-trained RNA foundation model of the kind described herein to perform different downstream tasks, including but not limited to examples described above. In this manner, the output of an RNA foundation model according to an embodiment can serve as a foundation for task-specific models.

A number of examples of downstream tasks have been presented to illustrate the power of an RNA foundation model for advancing understanding of RNA structure, function, and evolution, which in turn can address real-world biological problems such as predicting virus evolution and regulating gene expression (e.g., in connection with treatment of diseases related to excesses or deficiencies in gene expression). It should be understood that an RNA foundation model of the kind described herein can be used to generate embeddings for any type of downstream task and that, for a particular downstream task, the embeddings generated by the RNA foundation model can be combined with other information about the RNA (e.g., the raw sequence and/or secondary structure information) obtained from other sources. In some embodiments, a pre-trained RNA foundation model can be fine-tuned for a specific downstream task, e.g., by joint training of the RNA foundation model and another deep learning model associated with the downstream task, or by providing a transformer or other layer in the downstream model to fine-tune the RNA-FM embeddings in connection with a specific downstream task. However, such fine-tuning is not required; examples above illustrate how embeddings from a pre-trained RNA foundation model can improve the performance of downstream models in the absence of fine-tuning.

In various embodiments, different operations described above (e.g., training the RNA foundation model and training the downstream task(s)) can be performed in the same computer system or in different computer systems, with the computer system that performs the downstream task accessing the RNA foundation model via a local-area or wide-area network (e.g., using a client/server interaction protocol with the client sending queries to a server that hots the RNA foundation model). It should be understood that a computer system can include hardware components of generally conventional design (e.g., processors, memory and/or other storage devices, user interface components, network interface components) and that program code or other instructions can be provided to the computer system to cause the system to perform computations and/or other processes implementing embodiments described herein or aspects thereof.

Techniques described herein can be implemented by suitable programming of general-purpose computers. A general-purpose computer can include a programmable processor (e.g., one or more microprocessors including a central processing unit (CPU) and one or more co-processors such as graphics processing units (GPUs), or other co-processors optimized to implement nodes of a deep neural network) and memory to store instructions and data used by the programmable processor. A general-purpose computer can also include user interface components such as a display, speakers, keyboard or keypad, mouse, touch pad, track pad, joystick, touch screen, microphone, etc. A general-purpose computer can also include data communication interfaces to transmit data to other computer systems and/or receive data from other computer systems; examples include USB ports; Ethernet ports; other communication ports to which electrical and/or optical signal wires can be connected; and/or antennas and supporting circuitry to implement wireless communication protocols such as Wi-Fi, Bluetooth, NFC (near-field communication), or the like. In some embodiments, a computer system includes a single computer apparatus, where various subsystems can be components of the computer apparatus. The computer apparatus can have a variety of form factors including, e.g., a laptop or tablet computer, a desktop computer, etc. A computer system may include a monitor, printer or other suitable display for providing any of the results mentioned herein to a user. In other embodiments, a computer system can include multiple computer apparatuses, each being a subsystem, with internal components. A computer system can include a plurality of components or subsystems, e.g., connected together by external interface or by an internal interface. In some embodiments, computer systems, subsystems, or apparatuses can communicate over a network. For instance, a computer system can include a server with massive processing power to implement deep neural networks and a client that communicates with the server, providing instructions for specific network structures and operations.

It should be understood that any of the embodiments of the present invention can be implemented in the form of control logic using hardware (e.g., an application specific integrated circuit or field programmable gate array) and/or using computer software with a programmable processor in a modular or integrated manner. As used herein a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using a programming platform such as MATLAB, or any suitable computer language such as, for example, Java, C, C++, C#, Objective-C, Rust, Golang, Swift, or scripting language such as Perl, Python, or PyTorch, using, for example, conventional or object-oriented techniques. The software code may be stored as a series of instructions or commands on a computer readable storage medium; suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable storage medium may be any combination of such storage devices or other storage devices capable of retaining stored data. Computer readable storage media encoded with the program code may be packaged with a compatible device or provided separately from other devices. Any such computer readable storage medium may reside on or within a single computer product (e.g. a hard drive, a CD, or an entire computer system), and may be present on or within different computer products within a system or network.

Such programs may also be encoded and transmitted using carrier signals adapted for transmission via wired, optical, and/or wireless networks conforming to a variety of protocols, including the Internet. As such, a computer readable transmission medium (which is distinct from a computer readable storage medium) may be created using a data signal encoded with such programs.

Any of the methods described herein may be totally or partially performed with a computer system including one or more processors, which can be configured to perform the steps. Thus, embodiments can involve computer systems configured to perform the steps of any of the methods described herein, potentially with different components performing a respective step or a respective group of steps. Although presented as numbered steps, steps of methods herein can be performed at a same time or in a different order. Additionally, portions of these steps may be used with portions of other steps from other methods. Also, all or portions of a step may be optional. Additionally, and of the steps of any of the methods can be performed with modules, circuits, or other means for performing these steps.

The specific details of particular embodiments may be combined in any suitable manner without departing from the spirit and scope of embodiments of the invention. However, other embodiments of the invention may be involve specific embodiments relating to each individual aspect, or specific combinations of these individual aspects.

The above description is illustrative and is not restrictive. Many variations of the invention will become apparent to those skilled in the art upon review of the disclosure. The scope of patent protection should, therefore, be determined not with reference to the above description, but instead should be determined with reference to the following claims along with their full scope or equivalents.

What is claimed is:

1. A computer-implemented method comprising:
obtaining a large-scale training dataset of RNA sequences including unannotated RNA sequences;
training an RNA foundation model using the large-scale training dataset, wherein the RNA foundation model includes a plurality of transformer encoder blocks that produce an output embedding corresponding to an input RNA sequence; and
providing a query interface to the trained RNA foundation model, wherein the query interface receives a query RNA sequence and produces a corresponding output embedding.

2. The method of claim 1 wherein the RNA foundation model includes an initial embedding layer that embeds each nucleotide token into a high-dimensional vector.

3. The method of claim 1 wherein the training of the RNA foundation model is performed using a self-supervised training process.

4. The method of claim 3 wherein the self-supervised training process includes:
randomly replacing a fraction of original nucleotide tokens in a first RNA sequence from the large-scale training dataset with either a mask token or a randomly-selected nucleotide token to produce a masked sequence;
using the RNA foundation model to generate an output embedding for the masked sequence; and
predicting, based on the output embedding for the masked sequence, which original nucleotide token corresponds to a particular mask token in the masked sequence.

5. The method of claim 4 wherein the self-supervised training process further includes:
computing a cross-entropy loss based at least in part on the prediction.

6. The method of claim 1 wherein obtaining the large-scale training dataset includes:
obtaining an initial dataset of RNA sequences; and
preprocessing the initial dataset of RNA sequences to obtain the large-scale training dataset, wherein preprocessing includes standardizing nucleotide tokens and removing duplicate RNA sequences.

7. The method of claim 1 further comprising:
training a task-specific downstream system to predict a structural or functional characteristic of an input RNA sequence, the task-specific downstream system including a module that uses the query interface of the trained RNA foundation model to obtain an output embedding corresponding to the input RNA sequence and a machine-learning module that uses the input RNA sequence and the corresponding output embedding as inputs.

8. The method of claim 7 wherein the training of the task-specific downstream system includes a supervised training process.

9. The method of claim 7 wherein the task-specific downstream system is trained to predict secondary structure of a given RNA sequence.

10. The method of claim 7 wherein a plurality of different task-specific downstream systems are trained to predict different structural or functional characteristics and wherein all of the task-specific downstream systems obtain output embeddings from the same trained RNA foundation model.

11. The method of claim 1 wherein the RNA sequences in the large-scale training dataset are non-coding RNA sequences.

12. A computer-implemented method comprising:
obtaining, for each of a plurality of RNA sequences, a corresponding output embedding from an RNA foundation model that includes a plurality of transformer encoder blocks and that has been pre-trained to produce an output embedding corresponding to an input RNA sequence using an unsupervised learning process;
training a task-specific machine-learning model to predict a structural or functional characteristic of an input RNA sequence using a supervised learning process with annotated training data, wherein the task-specific machine-learning model uses as input a combination of the input RNA sequence and the corresponding output embedding produced by the RNA foundation model; and
using the trained task-specific machine-learning model to make a prediction for a testing input RNA sequence.

13. The method of claim 12 wherein the task-specific machine-learning model is trained to predict secondary structure of the input RNA sequence.

14. The method of claim 13 wherein the task-specific machine-learning model is a residual network.

15. The method of claim 12 wherein the RNA foundation model is trained using only non-coding RNA sequences.

16. The method of claim 12 wherein the task-specific machine-learning model is trained to predict a protein-RNA interaction of the input RNA sequence.

17. The method of claim 12 wherein the task-specific machine-learning model is trained to predict a parameter related to a gene expression regulation function of the input RNA sequence.

18. A system comprising:
a memory to store an RNA foundation model that includes a plurality of transformer encoder blocks and that has been pre-trained to produce an output embedding corresponding to an input RNA sequence using an unsupervised learning process;
an interface to receive queries from one or more requesting systems, each query including a queried RNA sequence; and
a processor coupled to the interface and the memory, the processor being configured to:
input the queried RNA sequence into the RNA foundation model to obtain a corresponding output embedding; and
return the output embedding to the requesting system via the interface.

19. The system of claim 18 wherein the processor is further configured to perform training of the RNA foundation model.

20. The system of claim 19 wherein the processor is further configured such that performing training of the RNA foundation model includes:
randomly replacing a fraction of original nucleotide tokens in a first RNA sequence from a training dataset with either a mask token or a randomly-selected nucleotide token to produce a masked sequence;
using the RNA foundation model to generate an output embedding for the masked sequence; and
predicting, based on the output embedding for the masked sequence, which original nucleotide token corresponds to a particular mask token in the masked sequence.

21. The system of claim 18 wherein the requesting system is configured to use the output embedding in a machine-learning task that predicts a structural or functional characteristic of the queried RNA sequence based at least in part on the output embedding.
